(19) United States
(12) Patent Application Publication
(10) Pub. No.: US 2009/0041792 A1
(43) Pub. Date: Feb. 12, 2009

(54) Dendritic cells, uses therefor, and vaccines and methods comprising the same

(75) Inventors: Filippo Belardelli, Rome (IT); Maria Ferrantini, Rome (IT); Caterina Lapenta, Florence (IT); Laura Lattanzi, Rome (IT); Stefania Parlato, Rome (IT); Stefano Maria Santini, Rome (IT)

Correspondence Address: QUINE INTELLECTUAL PROPERTY LAW GROUP, P.C., P O BOX 458, ALAMEDA, CA, 94501, US

(73) Assignee: Istituto Superiore di Sanita, Roma (IT)

(21) Appl. No.: 11/880,443

(22) Filed: Jul. 19, 2007

(51) Int. Cl. A61K 45/00; C12N 5/06; A61P 37/04

(52) U.S. Cl. 424/184.1; 435/377

(57) ABSTRACT

Provided is a method of cross-priming CD8+ T cells to antigens using Dendritic Cells cultured in the presence of a type I Interferon and GM-CSF, and vaccines and methods of vaccination comprising said Dendritic Cells.

### FIELD OF THE INVENTION

The present invention relates to methods of cross-priming CD8+ T cells to antigens using Dendritic Cells cultured in the presence of a type I Interferon and GM-CSF, and vaccines and methods of vaccination comprising said Dendritic Cells.

### INTRODUCTION

Dendritic cells (DCs) are considered the most potent antigen presenting cells (APCs), that play a crucial role in the stimulation of primary and secondary CD4+ and CD8+ T cell responses. Immature dendritic cells are characterized by efficient phagocytic activity that allows antigen up-take and processing. During the maturation process, DCs become less efficient in antigen capturing and processing but more specialized in presenting immunogenic peptides and in activating naïve T cells. DCs maturation can be mediated by inflammatory cytokines, or by additional stimuli such as CD40L, LPS or virus infection. All these stimuli can trigger up-regulation of MHC class I antigen-processing machinery as well as of costimulatory molecules (CD40, CD80, CD86) and DC maturation marker CD83 necessary for T-cell activation.

During a viral infection or a malignant transformation, dendritic cells acquire antigens from the affected sites, migrate to the lymph node and present peptides associated to MHC class I molecules, to CD8+ T cells. The mechanism by which DCs phagocytose exogenous antigens from the extracellular environment and efficiently present peptides associated to MHC class I molecules, to CD8+ T cells, is called cross-presentation and is likely the most important mechanism for the priming of CD8+ T cells responses against exogenous antigens in vitro and in vivo [1, 2]. Immune responses are regulated by signals associated with infection.

One host-derived infection-associated signal that stimulates cross-priming is type I IFN (IFN α-β). IFN α-β is expressed rapidly by cells in response to viral infection and it also shows a crucial role in linking innate and adaptive immunity [3]. In particular, it has been shown that IFN-α can efficiently promote the cross-priming of CD8+ T cells in mouse models [2].

Depending on their state of maturation, dendritic cells can cross-prime or cross-tolerize T cells [4]. DCs need to receive an activation signal to become competent to induce cross-priming, a process called “licensing” of APC [5]. Cross-presentation of antigens by “unlicensed” DCs stimulate an abortive response that culminates in cross-tolerance [6].

It has been reported that only mature DCs, such as those obtained from culturing immature GM-CSF/IL-4 DCs with tumor necrosis factor [symbol] (TNF-α) and prostaglandin E₂ (PGE₂), are efficient antigen presenting cells (APCs) for cross-priming of exogenous antigens to CD8+ T cells [7, 8]. Previous studies demonstrated that DCs generated from human monocytes after a single-step 3-days culture in the presence of IFN- [symbol] and GM-CSF, exhibit phenotypic and functional properties typical of activated partially mature DCs [9] and are more efficient than immature IL-4-DCs, in inducing a Th-1 type immune response and CD8+ T cells response against defined antigens in different model systems [10-13].

The priming and expansion of antigen-specific CD8⁺ T cell response is a complex process involving concerted interactions between lymphocytes and dendritic cells, the professional antigen-presenting cells playing a pivotal role in linking innate and adaptive immunity [1, 2]. The priming of antigen-specific CD8⁺ T cells requires recognition through the T cell receptor of peptide-MHC class I complexes on the surface of appropriate APCs. This event occurs when viral proteins are synthesized within an infected cell, where cytoplasmic proteasomes and peptidases degrade them into peptides, which are then translocated into the endoplasmic reticulum for the access to newly formed MHC class I molecules and transport to the cell surface.

However, suitable peptides may also be derived from exogenous antigens intersecting this pathway after endocytosis by APCs, in the cross-presentation process. As mentioned above, DCs must undergo a special activation process or “licensing” step in order to cross-prime CD8⁺ T cells. Under pathological conditions, DCs are “licensed” by engagement of surface CD40 by activated CD4⁺ helper T cells or by microbe-derived macromolecules, which can trigger DC maturation and up-regulate the expression of surface co-stimulatory molecules.

It is generally assumed that only mature DCs can efficiently induce cross-priming of CD8⁺ T cells against exogenous antigens [3, 4]. In considering the events leading to the generation of mature DCs from monocytes, the vision is generally influenced by the widely used two-step culture protocol: i) immature DCs are generated as a result of several days of culture in the presence of GM-CSF/IL-4; ii) a second culture step in the presence of maturation factors is required to obtain mature DCs [3, 5].

We previously demonstrated that highly active partially mature DCs are generated from monocytes after a single step of 3-day culture with IFN-α/GM-CSF (IFN-DCs) [6]. However, the mechanisms underlying this special attitude of IFN-DCs was unclear. Thus, the state of the art is that only mature DCs, such as those obtained from culturing immature GM-CSF/IL-4 DCs with tumor necrosis factor [symbol] (TNF-α) and prostaglandin E₂ (PGE₂), are capable of efficiently presenting antigens cross-priming CD8+ T cells. On the whole, it is generally thought that only mature DCs can efficiently prime T cells. DC can be matured by different methods known in the literature and laboratory practice, such as exposure to a cytokine cocktail containing TNF-α, IL-1β, IL-6 and PGE2, or treatment with sCD40L, addition of LPS as well as other bacteria-derived molecules and so forth.

Surprisingly, we found that IFN-conditioned DCs (IFN-DCs) are licensed for efficient CD8⁺ T cell priming, independent of CD4. What was particularly surprising was that the IFN-DCs couple a significant phagocytic activity (typical of immature DCs) with a particularly strong efficiency of “cross-priming” (superior to that of bona fide mature DCs). Thus, not only have we shown that IFN-DCs are capable of stimulating CD8+ expansion following presentation of an antigen, we have also shown that they are more efficient at doing so than mature DCs such as IL-4 DCs, and that this is licensing can be achieved in the absence of CD40 Ligand.

In fact, the differentiation/activation pathway of IFN-DCs resembles that of DCs rapidly generated after in vivo exposure of monocytes to infection-induced cytokines. DC's cultured and matured as taught in the art are often referred to herein as IL4-DCs as they are matured by exposure to IL-4.

What was also surprising was that viral antigens, even at low concentrations, are more efficiently cross-presented to CD8⁺ T cells by IFN-DCs compared to IL4-DCs. This is despite that the fact that antigen uptake and antigen processing capabilities were comparable. We also found that the IFN-DCs can be matured in the absence of the CD40 Ligand (CD40L).

As mentioned above, IFN-DCs are more efficient than CD40L-matured IL-4-DCs (mIL-4-DCs) in inducing a CD8⁺ T cell response in mice. Furthermore, IFN-DCs were much more efficient than mIL-4-DCs in inducing cross-priming of CD8⁺ T cells against HIV antigens.

Of note, upon CD40-CD40L interaction, IFN-DCs up-regulate IL-23 and IL-27 subunit transcripts to a higher extent than IL-4-DCs.

We also found that IFN-DC exhibit increased expression of selected Scavenger Receptors (SRs), among them LOX-1, and efficiently present exogenous molecules stimulating strong T cell responses.

### SUMMARY OF THE INVENTION

Thus, in a first aspect the present invention provides a method of inducing a CD8⁺ T cell response to an antigenic peptide, comprising:

culturing a monocytic cell in the presence of a Type I Interferon, Granulocyte-Mcrophage Clony-Simulating Factor (GM-CSF) and an antigen, to provide a cultured dendritic cell which presents said peptide complexed with an MHC class I molecule on its cell surface, and

exposing the cultured dendritic cell to a population of naïve CD8+ T cells.

It will be appreciated that the naïve CD8+ T cells each express a different T cell receptor, specific for an antigen. If the T cell receptor on a CD8+ T cell recognizes the antigen presented to it, when the CD8+ T cell is exposed to an Antigen Presenting Cell (APC), such as the Interferon cultured cell above, the CD8+ T cell will undergo clonal expansion, triggering an immune response against that antigen. This is readily detectable by methods known in the art, including ELISPOT assays and in vitro cytotoxicity assays.

In some embodiments, the method comprises stimulating expansion of a CD8⁺ T cell or eliciting a CD8⁺ T cell response to the antigenic peptide. In some embodiments, the method comprises cross-priming of CD8⁺ T cells to the antigenic peptide. In some embodiments, this includes inducing clonal expansion of the CD8⁺ T cell following contact with the MHC class I-peptide complex, and recognition of said peptide complex by the T cell receptor on the naïve CD8⁺ T cell. In some embodiments, the CD8⁺ T cell is a naïve CD8⁺ T cell, in other words a CD8⁺ T cell that has not yet been the subject of clonal expansion brought about by recognition of antigenic peptide complex on an antigen presenting cell (APC) by the T cell receptor.

In some embodiments, the dendritic cell is an “Interferon-Dendritic Cell” (IFN-DC), as taught in the present application and according to Santini et al (Journal of Experimental Medicine 2000, Vol. 191, pages 1777 to 1788), which is hereby incorporated by reference. The terms “cultured dendritic cell” and IFN-DC are used interchangeably herein.

The cultured dendritic cell is obtainable by culturing a monocyte in the presence of Interferon and GM-CSF, thereby providing the IFN-DC. A classical immature dendritic cell, for instance one cultured in the presence of IL-4 and GM-CSF, is thereby distinct from an Ifn-DC, cultured in the presence of a type I IFN and GM-CSF, as a classical immature dendritic cell is already committed along a different differentiation pathway. Indeed, the cultured dendritic cell is not a fully matured dendritic cell, for instance that obtainable by culturing a monocyte in the presence of Interleukin-4 (IL-4) and GM-CSF. Monocytes differentiate into dendritic cells in the presence of both IL-4 and GM-CSF. IL-4 is necessary but not sufficient to drive monocyte differentiation into mature DCs (IL-4 DCs), but this is a different path way and hence a different cell from an IFN-DC.

The interferon is a type I interferon. In some embodiments, the interferon is IFN-alpha (IFNα). In other embodiments, the interferon is interferon-beta (IFNβ). Non-human equivalents will be readily apparent to the skilled person, where required.

Granulocyte-Mcrophage Clony-Simulating Factor, often abbreviated to GM-CSF, is a protein secreted by macrophages, T cells, mast cells, endothelial cells and fibroblasts. GM-CSF is a cytokine that functions as a white blood cell growth factor. GM-CSF stimulates stem cells to produce granulocytes (neutrophils, eosinophils, and basophils) and monocytes. Monocytes exit the circulation and migrate into tissue, where upon they mature into macrophages. It is thus part of the immune/inflammatory cascade, whereby activation of a small number of macrophages can rapidly lead to an increase in their numbers, a process crucial for fighting infection. Non-human equivalents will be readily apparent to the skilled person, where required.

It will be understood that, in some embodiments, the step of culturing the cell comprises contacting the cell with interferon. Suitable conditions are described in Santini et al (supra). In some embodiments, the cultured dendritic cell, i.e. the IFN-DC, is characterised by the cell surface markers expressed thereon. In some embodiments, these include at least one of the following BDCA2, CD123, thereby giving the dendritic cells a phenotype equivalent to matured DCs, and in some embodiments, to CD123⁺ BDCA2⁺-plasmacytoid dendritic cells. In some embodiments, the IFN-DCs do not express BDCA1, so the IFN-DCs are BDCA1⁻. In some embodiments, the IFN-DCs are characterised by up-regulation of at least one of the following: CD40, CD80 and DC86. In some embodiments, the IFNDCs comprise at least one and, in some embodiments, all three (CD40⁺, CD80⁺ and DC86⁺), of these proteins expressed on their cell surface. Furthermore, in some embodiments, the expression of these proteins is also associated with the expression of the dendritic cell maturation marker CD83 (CD83⁺), although this can also be found in classically matured DCs, such as IL-4 DCs.

The IFN-DCs, in some embodiments, may also be characterised by the fact that HSP70 recognition by IFN-DCs is inhibited by the presence of anti-HSP70 monoclonal antibody (anti-HSB70 mAb). This is in contrast to dendritic cells matured in the presence of interleukins, such as IL4, for instance, where recognition of HSB70 is not affected by the presence of the anti-HSP-70 monoclonal antibody.

In some embodiments, the IFN-DCs are capable of inducing a strong TH1 immune response, together with a CD8⁺ T cell response against the antigen. In some embodiments, these cells show an increased capability to induce cross-priming of CD8⁺ T cells against said antigen.

In some embodiments, the antigen is autologous, being from the same individual. In other embodiments, the antigen is allogeneic, being from an other individual of the same species having a different allele at the same genetic locus. In other embodiments, the antigen is exogenous, being derivable from viruses, in particular, as described below.

In some embodiments, the IFN-DCs exhibit increased expression of the proteasome regulator sub unit PA28alpha (PA28α). In some embodiments, the IFN-DCs show increased expression of the catalytic sub units of said proteasome. In some embodiments, the IFN-DCs show both of these properties, it will be understood that this is in comparison to mature DCs, as obtainable by contacting monocytes with IL4 (so-called ILA-DCs).

In some embodiments, the method occurs in vitro and the expanded CD8⁺ T cells are introduced into the patient. In some embodiments, the naïve or unexpanded CD8⁺ T cells have first been removed from the patient and are subsequently reintroduced to the same patient. It is also envisaged that a progenitor of the naïve CD8⁺ T cells, such as stem cells, can be used in the present method to provide suitable CD8⁺ T cells.

In some embodiments, the patient is a mammal, including mice and primates. In some embodiments, the patient is a human.

It will be appreciated that reference to MHC molecules also include the human equivalent, HLA molecules, which serve the same function in humans. Accordingly, in some embodiments, the peptide forms a complex on the dendritic cell with HLA class I molecules. In some embodiments, the HLA molecules are Class I HLA haplotypes, so that the CD8+ T cells are restricted CD8+ T cells or are capable of recognising said HLA type. In some embodiments, the HLA molecules are of the haplotype HLA-A. In some embodiments, the HLA molecules are of the haplotype HLA-A2. In some embodiments, the HLA molecules are of the haplotype HLA-A2.1. In other embodiments, the HLA molecules are haplotypes selected from: HLA-A1, -A3, -A24, -A29, -A31 or -A33. In other embodiments, the HLA molecules are haplotypes selected from: HLA-B, -C, -E, -F and -G.

In some embodiments, the dendritic cell is obtained by culturing a monocyte in the presence of interferon and GM-CSF. In some embodiments, the dendritic cell is obtained by the method taught in Santini et al 2000 (supra).

In some embodiments, the type I interferon and the GM-CSF may be provided one after the other. In other embodiments, the type I interferon and the GM-CSF may be provided at the same time.

In some embodiments, the antigen is added before, during or after the monocyte is exposed to interferon and/or GM-CSF. In some embodiments the monocyte are co-cultured in the presence of interferon, together with the antigen or a source of antigens. However, in other embodiments, the cultured dendritic cell, having been pulsed with the interferon and/or GM-CSF is then contacted with the antigen or source of antigens. This allows the increased phagocytic activity of the IFNDC 4 antigens to be harnessed.

The antigen may be any antigen that elicits a CD8+ immune response. For instance, this may include an antigen derived from an exogenous source, particularly a virus. In some embodiments, the virus is HIV and in some of these embodiments, the antigen is derived from the expression products of gag, pol, env (gp160, gp140, gp120), and nef, such as peptides or the full protein sequences, provided that these comprise an epitope. In some embodiments, the antigen is a tumor-associated antigen (TAA). These include Epstein Barr Virus (EBV), including sub-dominant epitopes such as the LMP-2 epitope of EBV, Hepatitis viruses, especially the Hepatitis C Virus (HCV), including the NS3 peptide, the E6 and E7 proteins from HPV (Human Papillomavirus), tumoral antigens. In some embodiments, the tunoural antigens are selected from the group consisting of: especially those associated with cervical carcinoma, prostatic cancer, renal and lung cancer, and melanoma.

A population of CD8⁺ T cells can be found in vivo in a lymph node, for instance, or in vitro, as will be apparent. In some embodiments, the population can consist of as little as one naïve or unexpanded CD8⁺ T cell. However, in other some embodiments, the population consists of at least one hundred or one thousand such CD8⁺ T cells.

Also provided is a method of inducing a CD8⁺ T cell response to an antigenic peptide, comprising contacting a dendritic cell, which presents said peptide complexed with an MHC class I molecule, with a CD8+ T cell capable of recognizing said peptide-MHC class I complex, wherein the dendritic cell is obtainable by culturing a monocyte in the presence of Interferon and GM-CSF.

The invention also provides is a method of inducing a CD8⁺ T cell response to an antigenic peptide, comprising contacting a dendritic cell with a CD8+ T cell,

the antigenic peptide being presented in a complex with an MHC class I molecule, or its equivalent, on the surface of the dendritic cell, and

the CD8+ T cell comprising a T cell receptor capable of recognizing said peptide-MHC class I complex, wherein

the dendritic cell is obtainable by culturing a monocyte in the presence of a type I interferon and GM-CSF.

Also provided is a vaccine for an antigen comprising the IFN-DCs presenting an antigenic peptide and adapted for suitable administration to allow recognition of said antigen by the T cell receptor of CD8⁺ T cells. In some embodiments, the vaccine may be administered intravenously, subdermally, intramusculuarly, transmucosally, transdermally, intranodal injection or in the form of a patch or spray, for instance.

Also provided is a method of vaccination comprising administering the vaccine to a patient. In some embodiments, the antigen is obtained from the patient, for instance, by a blood sample or tissue extract, and contacted with the dendritic cell, thereby allowing the presentation of the antigen, or a fragment thereof, on the surface of the dendritic cell in complex with the MHC class 1 molecule, the dendritic cell (comprising said complex) being reintroduced into the patient, in the form of a vaccine, as described above. Suitable vaccination protocols will be apparent to the skilled person in light of the disease or virus to be combated.

We have shown that Dendritic cells (DCs) generated after a short-term exposure of monocytes to IFN-alpha and GM-CSF (IFN-DCs) are highly effective in inducing cross-priming of CD8⁺ T cells against viral antigens. We have further investigated the mechanisms responsible for the special attitude of these DCs and compared their activity with that of reference DCs. Antigen uptake and endosomal processing capabilities were similar for IFN-DCs and IL-4-derived DCs.

Both DC types efficiently cross-presented soluble HCV NS3 protein to the specific CD8⁺ T cell clone, even though IFN-DCs were superior in cross-presenting low amounts of viral antigens. Moreover, when DCs were pulsed with inactivated HIV-1 and injected into hu-PBL-SCID mice, the generation of virus-specific CD8⁺ T cells was markedly higher in animals immunized with IFN-DCs than in mice immunized with CD40L-matured IL-4-DCs. Surprisingly, in experiments with purified CD8⁺ T cells, IFN-DCs were superior with respect to CD40L-matured IL-4-DCs in inducing in vitro cross-priming of HIV-specific CD8⁺ T cells. This property correlated with enhanced potential to express the specific subunits of the IL-23 and IL-27 cytokines. These results suggest that IFN-DCs are directly licensed for an efficient CD8⁺ T cell priming by mechanisms likely involving enhanced antigen presentation and special attitude to produce IL-12 family cytokines.

DNA microarray technology was then used to get more insights on the molecular mechanisms activated by IFN-α during the DC activation/differentiation process. We performed global transcript analysis in IFN-DCs compared to monocytes treated with GM-CSF alone and to DCs generated with GM-CSF and IL-4 by using Affymetrix platform. The analysis of transcriptional profiles showed that IL-4 treatment mainly induced genes related to metabolic pathways, on the contrary a 3-day IFN-alpha treatment of human monocytes induced an over-expression of genes involved in immunological pathways, such as signal transducer activity, antigen processing and presentation, cytokine and chemokine activity. In particular, IFN-DCs showed a strong up-regulation of genes belonging to the Scavenger Receptor family. Among these, the main Hsp70 binding receptor LOX-1 was strongly induced following the IFN treatment, but LOX-1 expression was lost in completely mature dendritic cells. Moreover, binding experiments showed that using a neutralizing anti-LOX-1 mAb the Hsp70 binding to IFN-DCs was powerfully inhibited.

It was also surprising that LOX-1 is involved in apoptotic cell phagocytosis by IFN-DCs and in apoptotic bodies-derived antigens cross-presentation to purified CD8+ T. On the whole, our results indicate that IFN-DCs are characterized by a gene expression profile typical of highly activated mature DCs, and that the Hsp70 binding capacity of IFN-DCs is strongly dependent on LOX-1 expression.

We also evaluated the efficiency of IFN-DCs as compared to immature IL-4-DCs in the cross-presentation of EBV tumor-associated antigens. Firstly, we choose a completely autologous model system in which DCs from EBV-positive donors were loaded with apoptotic cells (apo-LCL) or cell lysates (lys-LCL) derived from autologous LCL, and then used as APCs for the stimulation of autologous PBMCs. Our results demonstrate that IFN-DCs loaded with a lysate of autologous LCL can efficiently expand a class II-restricted T cell response specific for autologous LCL, i.e. CD4+ T cells directed against EBV antigens. We report that IFN-DCs loaded with autologous apoptotic LCL could quite efficiently expand a class I-restricted T cell response specific for autologous LCL, therefore demonstrating the ability of IFN-DCs to cross-present EBV-derived TAAs to CD8+ T lymphocytes.

With regard to LOX-1 expression and its role in IFN-DCs, we have shown that:

i) LOX-1 is involved in the uptake of apoptotic cells at a significantly higher degree in the IFN-DCs as compared to the IL-4-DCs (see new FIG. 9); and

ii) LOX-1 mediates the cross-presentation by IFN-DCs of allogeneic apoptotic cell-derived antigens to autologous CD8+ T cells (see FIG. 10).

Finally, we also demonstrate that IFN-DCs are more potent than mature IL4-DCs in stimulating the cytotoxic activity of CTLs specific for a sub-dominant HLA-A2.1-restricted CD8+ epitope of the EBV LMP-2 antigen. However, the mechanisms underlying this special attitude of IFN-DCs to induce cross-presentation of exogenous antigens, remained to be determined. We report that, even though antigen uptake capacity appear to be similar in IFN-DCs and immature IL-4-DCs, nevertheless proteasomes from IFN-DCs exhibited an overall proteolytic activity higher than that exerted by proteasomes isolated from immature or LPS-treated IL-4-DCs. Moreover, it should be noted that IFN-DCs express higher amounts of PA28α, a proteasome activator that strongly increases the proteolytic activity of proteasomes [20].

The examination of the proteasome subunit expression in IFN-DCs compared to immature or mature IL-4-DCs, further support the mature phenotype exhibited by IFN-DCs. We have shown that:

i) the overall higher enzymatic activity of the proteasome in the IFN-DCs (see FIG. 15) as compared not only to immature IL-4-DCs, but also, surprisingly, to bona fide mature IL-4-DCs (EL-4-DCs treated with LPS); and

ii) that IFN-DCs loaded with apoptotic tumor cells (LCL) are more efficient than mature DCs in mediating the cross-presentation of a sub-dominant epitope of the EBV protein LMP-2 (that also represents a tumor-associated antigen in cells latently infected and transformed by EBV, such as the LCL) (see FIG. 14).

Where reference is made herein to the term “pulsing”, for instance in regard to pulsing monocytes in the presence of type I IFN, it should be understood that this covers “culturing” in the sense of culturing monocytes in the presence of type I IFN. The same also follows for the term “contacting,” which may be used interchangeably with “pulsing” or “culturing” in so far as this is in accordance with the present invention. This may be achieved, for instance as taught in Santini et al (Santini, S. M., Lapenta, C., Logozzi, M., Parlato, S., Spada, M., Di Pucchio, T. and Belardelli, F., Type I interferon as a powerful adjuvant for monocyte-derived dendritic cell development and activity in vitro and in Hu-PBL-SCID mice. J. Exp. Med. 2000. 191: 1777-1788).

### DESCRIPTION OF THE FIGURES

FIG. 1 As per Parlato et al. (2001, 98:3022-9), IFN-DCs expressed high levels of the lymphoid DC marker CD123 (IL-3Ra) which was poorly detected in IL-4-DCs. Notably, a remarkable percentage of IFN-DCs expressed the plasmacitoid marker BDCA2 which was undetectable in IL-4-DCs; on the contray, the IFN-DCs exhibited a marked reduction in the expression of BDCA1 myeloid marker, which was consistently expressed in IL-4-DCs.

FIG. 2 Phenotype, antigen uptake and antigen processing capacity by IFN-DCs and IL-4-DCs. (A) Percentage of DCs expressing a series of selected membrane markers as detected by FACS analysis. (B) Florescence intensity of selected membrane markers as detected by FACS analysis. Bars represent the percentage or the mean fluorescence intensity of cells expressing the selected membrane marker and the standard error. (C and D) Antigen uptake and processing by the IFN-DCs and IL-4 DCs. Cells were incubated for 60 min at 37° C. with 50 μg/ml of dextran-FITC conjugate (C) or 100 μg/ml of DQ-Ovalbumin (D). After 60 min, cells were washed and analysed by Flow cytometry. DQ ovalbumin is a self-quenched conjugate of albumin that exhibits bright green fluorescence only upon proteolityc degradation

FIG. 3. Comparative analysis of the ability of IFN-DCs and immature IL-4-DCs to phagocytose apoptotic LCL cells or LCL lysates. PHK67 green stained apoptotic LCL (A) or LCL lysates (B), were incubated with CD11c-PE labelled IFN-DC or immature IL-4-DC (ratio 2:1) for 4 hours. Controls were incubated at 4° C. for 4 hours (lower quadrants). After co-cultivation, the number of CD11c+-PHK67+ double-positive DCs was assessed by flow cytometry analysis. One representative experiment is shown.

FIG. 4. Differentially expressed genes by IFN- and IL-4-treatment.

Microarray experiments were performed by using Affymetrix HG U133A oligonucleotide arrays covering 14,500 well-characterized human genes. Significant Analysis Microarray (SAM) was performed to select the genes significantly modulated by the two treatments (IFN and IL-4) with respect to the common control (GM-CSF). We obtained a global list of 807 genes, significant for at least one of the two treatments. By crossing, for each treatments, the treatment/control (T/C) expression log ratios of all biological replicates, we obtained 32 T/C ratios that were assigned to the two groups (IFN and IL-4). The Two Class Unpaired problem was analysed with the SAM method. Thus, we obtained 140 “discriminant” genes: 73 genes whose T/C ratio was higher for IFN- than IL-4-treatment and 67 genes whose T/C ratio was higher for IL-4- than IFN-treatment. 645 genes were not differentially expressed in the two treatments.

FIG. 5. IFN-treatment up-regulates genes involved in phagocytic and endocytic processes.

Hierarchical clustering of the expression profiles of 73 discriminant genes modulated by IFN-treatment, as compared to ILA-treatment, is represented. Red and green colors indicate up- and down-regulation, respectively, in comparison to GM-CSF-treatment values (black).

FIG. 6. SRs expression in IFN-DCs.

A) The expression of some SR (CD14 and CD36) was evaluated by FACS analysis in IFN-DCs and IL-4-DCs; B) LOX-1 mRNA is only expressed in IFN-DCs. RT-PCR analysis showing the expression of LOX-1 in IFN-DCs, IL4-DCs and after their stimulation with LPS (1 ug/ml for 24 hrs). Data are representative of 3 experiments derived from different donors.

FIG. 7. Hsp70 binding neutralization in IFN-DCs by an anti-LOX-1 mAb.

50 ug/ml anti-LOX-1 mAb (gray shaded) or isotype control IgG1 mAb (dotted line) were added to IFN-DC and IL-4-DC cultures before incubation with 25 ng/ml Hsp70-FITC (bold line). Broken line corresponds to dendritic cells alone.

FIG. 8 Anti-LOX-1 mAb blocks the stimulation capability of Hsp70 in IFN-DCs. Hsp70 pre-treatment of IFN-DCs induced the proliferation of allogeneic lymphocytes in a similar manner with respect to the untreated IFN-DCs. On the contrary, the IFN-DC pre-treatment with Hsp70 induced a very strong proliferation of allogeneic PBMCs with respect to Hsp70 pre-treated-IL-4-DCs (data not shown). The presence of a neutralizing anti-LOX-1 mAb blocked the stimulation capability of Hsp70 in IFN-DCs, confirming the functional involvement of LOX-1 in the Hsp70-mediated activation of IFN-DCs.

FIG. 9. Phagocytosis assay by FACS analysis. The anti-LOX-1 mAb only inhibited the phagocytosis of apoptotic cells by IFN-DCs, whereas the pre-treatment of IL-4-DCs with the same neutralizing mAb did not affect their capability to phagocyte apoptotic debris.

FIG. 10. LOX-1 mediates the cross-presentation by IFN-DCs of allogeneic apoptotic cell-derived antigens to autologous CD8+ T cells. IFN- and IL-4-DCs were loaded with apoptotic cells and then co-cultured with autologous purified CD8+ T cells at different stimulator/responder ratios. The co-cultures were carried out in the presence or in absence of neutralizing anti-LOX-1 mAb. One representative out of three different experiments is shown.

FIG. 11. Expression level of the immunoproteasome subunits and of the proteins involved in the intracellular pathway of MHC class I antigen-processing machinery in IFN-DCs as compared to immature or 48 hours LPS-treated IL-4-DCs. Equal amount of proteins from total cell lysates were fractioned by SDS-PAGE, transferred onto nitrocellulose filters and probes with mAbs or polyclonal antisera specific for the a2 subunits, PA28a, LMP2, LMP7 and MECL-1 or TAP1, TAP2 and tapasin. All DC types expressed an equal amount of total proteasomes, as demonstrated by antibody specific for the constitutive a2 proteasome subunits. One representative experiment of six performed is shown.

FIG. 12 Presentation assay of the NS3-1406 peptide and cross-presentation of the whole NS3 protein to the specific CD8⁺ T cell clone (clone NS3-1). Cells (3×10⁴/test) of the CD8+ T cell clone NS3-1 specific for an HLA-A2 binding peptide HCV1406 were incubated, at a S/R cell ratio of 1:1.5 in a microculture plate, with IFN-DCs or IL-4-DCs previously loaded with (A) NS3 recombinant protein (50 μg, 10 μg, 1 μg or 0) or peptide HCV1406 (100 ng, 10 ng, 1 ng, 0.1 ng, 0.01 ng 0.001 ng/ml or 0) (C). After 18-h incubation at 37° C., cells were assayed for IFN-γ production by intracellular immunofluorescence staining followed by flow cytometry (see Materials and Methods for details). Each bar represents the mean (±SE) of values of three experiments. (B) Representative dot plot analysis of IFNγ expression by the CD8 clone NS3-1 stimulated with DCs loaded with the NS3 protein. (D) Representative dot plot analysis of IFNγ expression by the CD8 clone NS3-1 stimulated with DCs loaded with the NS3-1406 peptide.

FIG. 13. FIG. 13A: the preferential expansion of a class II-restricted T cell response specific for autologous LCL after PBMC stimulation with lys-LCL-loaded IFN-DCs was confirmed by a detailed analysis in ELISPOT assays of the specificity of the T cell line exhibiting the highest frequency of IFN-γ-secreting cells. FIG. 13B: in vitro expansion of autologous EBV-specific T cell lines by stimulation with DCs loaded with autologous apoptotic LCL cells. The figure shows the analysis of the response of T cell lines (1, 2, 3), generated after stimulation with IFN-DCs (left graph) or IL-4-DCs (right graph) loaded with autologous apoptotic LCL, evaluated in IFN-g ELISPOT assays performed on day 28 using as presenting cells autologous LCL (open bars) in the presence of anti-MHC class-I (filled bars) or MHC class-II (dotted bars) antibodies. Each bar represents the mean spot number of triplicates ±SD per 5×10³ responder cells.

The insert shows flow cytometric analysis of apoptotic LCL cells using Annexin V-FITC and PI staining. LCL cells were UV-B irradiated for 3′ and after 20 hours, stained with Annexin V-FITC/PI. The percentage of double positive annexin V-FITC+/PI+LCL (late apoptosis) obtained is >70%.

FIG. 14. Shows that when apo-LCL-loaded IFN-DCs were used as target cells of the CLG epitope-specific CTLs, a considerably higher level of specific lysis was obtained (70-80%) as compared to that reached against mature IL-4-DC counterparts (approximately 30-40%).

FIG. 15 shows the comparative analysis of the cleavage specificity of equal amounts of proteasomes semi-purified from IFN-DCs, immature IL-4-DCs, and IL-4-DCs treated with LPS for 20 hours. Both the tryptic-like (panel A) and postacidic-like (panel B) activities were augmented in proteasomes obtained from IFN-DCs as compared to both immature and LPS-treated IL-4-DCs, while the chymotryptic-like activity (panel C) was similar in IFN-DCs and LPS-treated IL-4-DCs and augmented with respect to that measured in proteasomes from immature IL-4-DCs. The expression levels of the immunoproteasome subunits was also evaluated in total cell lysates prepared from the same DC samples used for the analysis of the enzymatic activity (panel D).

FIG. 16 Comparative characterization of the expression of HIV-1 receptors and of the DC susceptibility to HIV infection.

Membrane expression of molecules involved in HIV entry and infection (A). Three days after HIV-1 infection, proviral load was analyzed in DCs by PCR for viral gag sequences (B). The sensitivity of the assay was tested by amplifying serial dilutions of DNA prepared from 8E5 cells which harbour one proviral copy/cell (B). Viral release from infected DCs was assessed by measuring the levels of the HIV-1 p24 protein in culture supernatants (C), as described in Materials and Methods.

FIG. 17 Phenotype and cytokine production by the different immature and mature DC types. (A) Representative dot histogram FACS® profiles of 4 types of DCs used in the in vivo experiments in the hu-PBL-SCID mouse model. (B) PGE₂ and cytokine levels in the culture supernatants of IFN-DCs, IL-4-DCs, mIFN-DCs and mIL4-DCs after their culture for 24 h in fresh medium. Each bar represents the mean concentration values (±SE) of three experiments.

FIG. 18

Generation of anti-HIV-1 antibodies in hu-PBL-SCID mice immunized with AT2-HIV-1-pulsed DCs ELISA detection of antibodies to the HIV-1 gp41 ectodomain epitope AVERY in the sera from hu-PBL-SCID mice immunized with virus-pulsed IFN-DCs or CD40L-matured IL-4-DCs (min-DC) as compared to the basal response in non-immunized hu-PBL-SCID mice (CTR). Three 10-fold serum dilutions (1:10 ▪; 1:100□; 1:1000□) from 3 mice in each group were tested. Each bar represents the mean (±SE) of values of 3 serum samples from individual mice.

FIG. 19

Generation of HIV-specific human CD8⁺ T cells in hu-PBL-SCID mice immunized with AT2-HIV-1-pulsed DCs. Elispot analysis of anti-HIV-1 CD8⁺ T cell response. Human cells recovered from three spleens of hu-PBL-SCID mice from each group were pooled. The assay was performed using as stimulators autologous AT2-HIV-1-pulsed or unpulsed DCs. Bars represent the CD8⁺ T cell response from hu-PBL-SCID mice immunized with either IFN-DCs or mIL-4-DCs (Exp. 1) and IFN-DCs or mIFN-DCs (Exp. 2), as compared to the basal CD8⁺ T cell response in non-immunized hu-PBL-SCID mice (CTR). Control cultures were incubated with unpulsed autologous DCs. The panel shows the results of one representative experiment out of three. Hu-PBL-SCID mice were immunized as described and Materials and Methods.

FIG. 20

In vitro cross-priming of CD8⁺ T cells against exogenous HIV-1 antigens by DCs co-cultivated with either total PBLs or purified CD8⁺ T cells. Purified CD8⁺ or total PBLs were stimulated on day 0 and restimulated on day 7 with the autologous IFN-DCs or mIL-4-DCs pulsed with AT-2-inactivated HIV-1 (stimulator/responder ratio of 1:4). Panel A shows the light scatter and dot plot analyses of the purified CD8⁺ T cell population used in the experiment illustrated in panels B and C. Control cultures were incubated with unpulsed autologous DCs. Exogenous IL-2 (25 U/ml) was added every 4 days. At day 14, the cultures were restimulated with DCs pulsed with AT2-HIV-1, before performing ELISPOT IFN-γ (B) and ELISPOT granzyme-B (C) assays, as described in Materials and Methods.
(D). Cytokine production in the supernatants of primary cultures stimulated three times with autologous DCs. Cytokines were measured as described in Materials and Methods. Data are representative of three experiments. No measurable levels of IL-2, IL-1β, IL-7, IL-18, IL-15 and TGFβ1 were detected.

FIG. 21 Evaluation of the levels of mRNA expression of the subunits of the IL-23 and IL-27 cytokines by TaqMan real-time RT-PCR analysis and of IL-12/IL-23 cytokine production by ELISA. (A) DCs were obtained from blood monocytes as described in Materials and Methods. Immature DCs were then induced to differentiate by overnight exposure to sCD40L. To measure cytokine mRNA expression, TaqMan real-time reverse transcriptase PCR (RT-PCR) analysis was used (Applied Biosystems, Foster City, Calif.). Total RNA was extracted from monocytes and DCs at different time points, and reverse transcription was carried out as previously described. TaqMan assays were performed according to the manufacturer's instructions with an ABI 7700 thermocycler (Applied Biosystems). PCR was performed, amplifying the target cDNA (p40, and p19 transcripts for IL-23. EBI-3 and p28 for IL-27), with β-actin cDNA as an endogenous control. Data was analyzed with the PE Relative Quantification software of Applied Biosystems. Specific mRNA transcript levels were expressed as fold increase over the basal condition (untreated monocytes). (B) IL-12 and IL-23 protein release in culture supernatant was tested by using a commercially available Elisa Kit. Each bar represents the mean concentration values (±SE) of three experiments.

### DESCRIPTION OF THE INVENTION

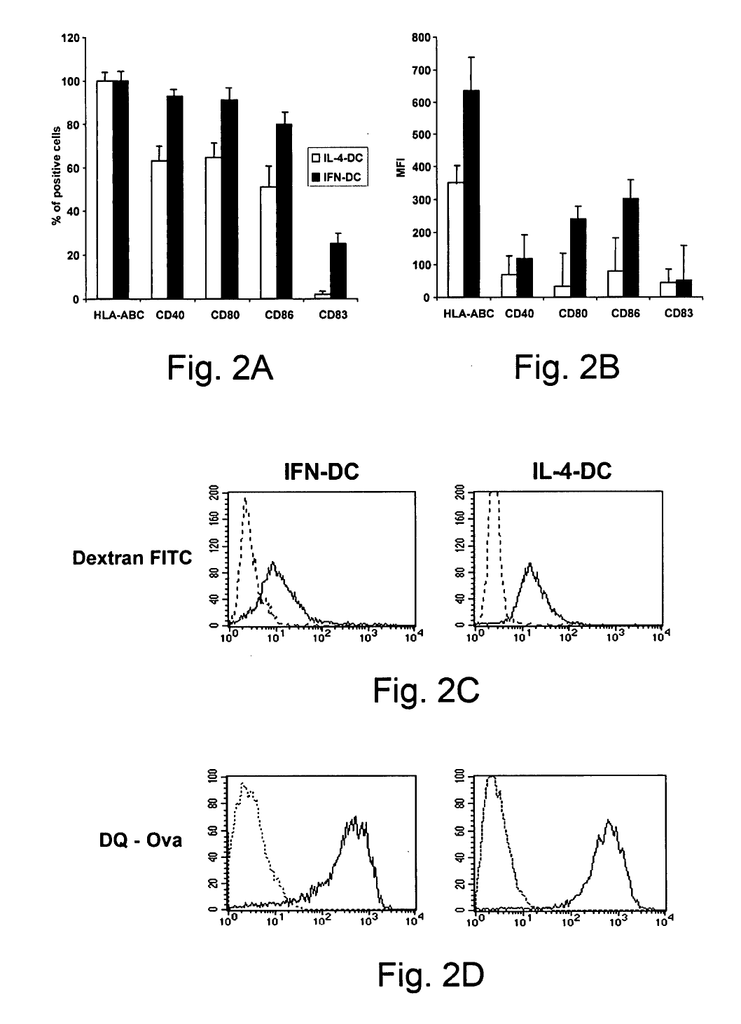

In the present study, we have shown that one single step culture of monocytes in the presence of IFN-α/GM-CSF is sufficient to generate DCs endowed with a special attitude for cross-priming of CD8⁺ T cells against exogenous antigens in vivo and in vitro, even in the absence of CD4⁺ T cell help. This special attitude to induce cross-priming of CD8⁺ T cells against exogenous antigens was not explained by increased antigen uptake and antigen processing capabilities, since these functions were comparable between the IFN-DCs and the immature IL-4-DCs (FIG. 2C, 2D).

Nevertheless, the IFN-DCs retained a superior attitude in cross-presenting low or limiting amounts of viral antigens to CD8⁺ T cells. Without being bound by theory it is thought that, since similar results were obtained with peptide pulsed DCs, it is likely that the higher levels of co-stimulatory and HLA class-I molecules expressed on IFN-DCs may explain this superior function, although we cannot rule out the possibility that the capacity of targeting antigens onto class I processing pathway is more efficient in IFN-DCs than in IL-4-DCs. However, this difference may, at least in part, be responsible for the enhanced capability of the IFN-DCs with respect to IL-4-DCs to induce an in vivo cross-priming of CD8⁺ T cells against HIV antigens in the hu-PBL-SCID model [7], even though other mechanisms, such as production of a special set of cytokines/chemokines by IFN-DCs could also play an important role.

It is generally believed that DC ability to activate and expand Ag-specific CD8⁺ T cells depends on the DC maturation stage and that DCs need to receive a “licensing” signal, associated with IL-12 production, in order to elicit cytolytic immune response. In particular, the provision of signals through CD40 Ligand-CD40 interactions on CD4⁺ T cells and DCs, respectively, is considered important for the DC licensing and induction of cytotoxic CD8⁺ T cells [14-16]. Although different stimuli can activate DCs, in our comparative studies with the IFN-DCs we have utilized mature DCs activated by CD40 ligation, which sustains prolonged NF-κB activation, high levels of IL-12 and effective CTL induction [14-18, 23, 24]. The finding that IFN-DCs were more effective than mIL-4-DCs in inducing cross-priming of CD8⁺ T cells against exogenous HIV antigens suggests that DC licensing for CD8⁺ T cell cross-priming can efficiently occur after a single-step short-term culture of monocytes in the presence of infection-induced cytokines (i.e., IFN-α).

In our experiments, the capability of the different DCs to induce cross-priming of CD8⁺ T cells against HIV antigens did not correlate with IL-12 production, at the time of DC injection into hu-PBL-SCID mice or before their co-culture with autologous T cells. In fact, as expected, the mIL-4-DCs utilized in our experiments produced large quantities of IL-12, while only low IL-12 levels were detected in supernatants from IFN-DCs (FIG. 17B). However, the finding that large amounts of IL-12 were secreted by IFN-DCs after contact with autologous lymphocytes indicates that these DCs are already committed to undergo terminal activation/maturation.

Interestingly, a further stimulation of IFN-DCs with sCD40L before in vivo immunization did not significantly enhance the capacity to stimulate CD8⁺ T cells, suggesting that the single IFN-α conditioning step was fully sufficient to directly generate “licensed” DCs. In our in vitro studies, we measured two types of cell response: i) CD8⁺ cells producing IFN-γ; ii) CD8⁺ cells releasing granzyme-B, which may represent cytotoxic effector cells.

Surprisingly, in the absence of CD4⁺T helper cells, IFN-DCs were superior with respect to mIL-4-DCs in inducing both types of CD8⁺ T cell responses. Interestingly, when the response of total PBLs stimulated with AT-2-HIV-pulsed DCs was studied, both IFN-DCs and mIL-4-DCs were equally capable to efficiently stimulate the expansion of IFN-γ-producing CD8⁺ T cells, while IFN-DCs were superior in the induction of granzyme-B-producing cells.

In this regard, it is worth mentioning that CTL effector diversity in terms of dissociated expression of granzyme-B and IFN-γ has been described [25]; most assays describe specificity and frequency of antigen-specific CD8⁺ cells rather than direct antiviral-effect [26], and IFN-γ-producing cells are mainly involved in macrophage activation and inflammation, while direct killing activity is associated with granzyme-B release. Our data showing that the granzyme-B-releasing CD8⁺ T cells are more effectively induced by IFN-DCs further emphasize the concept that distinct DC types can preferentially induce different CD8⁺ T cell subsets, which may differentially affect the quality of response.

There are several mechanisms by which IFN-α can influence the licensing of DCs, including the enhancement of expression of the peptide transporter TAP-1 [27], up-regulation of MHC class I antigens and induction of factors sustaining generation and activity of CD8⁺ T cells [28]. In particular, our results suggest a role of IL-23 and IL-27 in the Th1-promoting activity of IFN-DCs, as these cytokines appear to be produced at higher levels by the IFN-DCs and are important in enhancing IL-12-mediated CD8⁺ T cell responses [20, 21]. Noteworthy, the adjuvant activity of these cytokines has been demonstrated by a recent paper reporting an increase in the number of IFNγ-producing specific CD8⁺ cells upon administration of IL-23 and IL-27 [22]. Moreover, IL-23 has been shown to sustain CTL and Th1 immune responses to DNA immunization by increasing the rate of survival and proliferation [29].

Notably, IL-23 activity appears to be preferentially restricted to memory T cells, although it has also been demonstrated that IL-23 can synergize with IL-12 in promoting cytokine production by DCs themselves [30]. High levels of this cytokine could explain, at least in part, the better T cells response of IFN-DCs in terms of T cell IFN-γ production (FIGS. 19 and 20B).

On the other hand, IL-27 synergizes with IL-12 to induce IFN-γ production by naïve T cells and regulates IL-12 responsiveness of naïve CD4⁺ T cells through IL-12Rβ2 chain up-regulation [31, 32]. We suggest that an up-regulation of IL-27 production by the IFN-DCs could result in a higher response of naïve T cells to IL-12 action, thus leading to high levels of T cell IFN-γ secretion. We postulate that early exposure to IL-27, produced by IFN-DCs would commit naive T cells toward Th1 phenotype while exposure to IL-12 would favour subsequent expansion and stabilization of Th1 response with the contribution of IL-23 which had been shown to sustain the proliferation of memory T cells.

Recent studies have revealed the important role of type I IFN in linking innate and adaptive immunity [28, 33]. In particular, A. Le Bon and co-workers have shown that type I IFN-α can efficiently promote the cross-priming of CD8⁺ T cells in mouse models [34]. These results have led to the suggestion that virus-induced IFN can act as a major stimulus for vigorous generation of CD8⁺ T cell response, often observed in the course of some viral infections, by multiple mechanisms, including the promotion of cross-priming of CD8⁺ T cells against exogenous antigens [35].

Our results suggest that mechanisms similar to those described in mice [34, 35] can be operative in humans, supporting the concept that the in vivo generation of IFN-α-conditioned DCs represent a natural event required for an efficient in vivo cross-priming of CD8⁺ T cells against exogenous antigens in the course of infections. Lastly, our data may be relevant for the development of DC-based vaccines, which has recently emerged as an attractive strategy of therapeutic vaccination in patients with cancer and infectious diseases [3, 36, 37].

In fact, one critical issue for optimisation of DC-based vaccines is the identification of DCs endowed with functional features “optimal” for the induction of a protective anti-tumour response. While our results lead to a general attention to consider IFN-DCs as candidates for development of DC-based vaccines, our data also underline a specific interest for using IFN-α-conditioned DCs and AT-2-HIV as immunotherapy of HIV-1 infection. The interest on this strategy is, in fact, enhanced by a recently published report showing the efficacy of an AT-2-HIV-DCs vaccine in lowering HIV viremia in HIV-infected patients [38]. In view of this and of the several studies showing a special anti-HIV activity of IFN-DCs with respect to conventional DCs [6, 7, 13], the use of IFN-DCs in clinical trials of therapeutic vaccination of HIV-1 infected patients represents the natural direct extension of the present work.

### LOX-1 and IFN-DCs

Dendritic cells (DCs) are specialized phagocytes that plays an important role in clearance of infectious pathogens and dying host cells as a result of normal turnover of body tissues which yields apoptotic cells as well as infections causing tissue injuries and cell necrosis. Whereas phagocytosis of apoptotic debris occurring physiologically during the turn-over of a given tissue may be a means by which DC induce peripheral tolerance to self, the exposure to microbial products or allogeneic molecules carried by apoptotic bodies or necrotic cells promotes DC maturation and provides the stimulus to induce pro-inflammatory and immunostimulatory responses. In addition, upon capture of antigens (Ags), DC mature, increase antigen processing and presentation, and enhance migration to secondary lymphoid sites where they acquire the ability to activate specific CD4+ and CD8+ T lymphocytes initiating the adaptive immune response. DC maturation, characterized by enhanced expression of costimulatory molecules and secretion of immunoregulatory cytokines, can be triggered by direct interaction with pathogen products through pattern-recognition receptors (PRRs) such as Toll-like receptors (TLRs) and scavenger receptors (SRs), including LOX-1, or, alternatively, through cytokines produced by other infected cells such as type I interferon (IFN).

SRs are membrane endocytic receptors and mediate pathogen recognition in macrophages. Recently, several lines of evidence support a role of SRs in the phagocytic and antigen-presentation functions of DC. Like other SR family molecules, LOX-1 recognizes diverse pathophysiological ligands, including oxidized low density lipoprotein (0×LDL), aged/apoptotic cells, activated platelet and bacteria. LOX-1 was originally detected on endothelial cells and has been implicated in endothelial activation and vascular dysfunction associated with the initial steps in the process of atherogenesis and inflammation during atherosclerosis. In fact, the expression of LOX-1 is highly inducible by proinflammatory stimuli, including tumor necrosis factor (TNF)-a, lipipolysaccharide (LPS) and transforming growth factor (TGF)-b. In addition, LOX-1 has been associated with other functions related to immunity including leukocyte adhesion as the tethering receptor responsible for leukocyte adhesion rolling on endothelial cells. Remarkably, LOX-1 has been found to be the main receptor expressed on human DC mediating heat shock protein (HSP)-binding and antigen cross-presentation. In particular, this SR seems to play a pivotal role in the process by which some exogenous molecules such as HSP or apoptotic bodies are endocytosed by DC, gain access to the MHC class I pathway and stimulate CTL responses.

In most cases, human DCs for clinical studies are generated from peripheral blood monocytes by 4 to 7 days of incubation with high concentrations of interleukin (IL)-4 and granulocyte-macrophage-colony-stimulating factor (GM-CSF), that is IL-4-DC, matured with one of many possible maturation stimuli such as CD40L. We have shown that DCs generated ex vivo from human monocytes in the presence of IFN-{alpha} and GM-CSF, namely IFN-DC, are highly active partially mature antigen-presenting cells (APCs) with markedly enhance dendritic cell activities. In fact, in contrast to IL-4-DC, IFN-DC express high levels of CD1a, CD1c, Class I and II major histocompatibility molecules, CD80, CD83, and CD86. Functionally, IFN-DCs are highly active in inducing a Th-1 type of immune response and CD8+ T cell responses against defined antigens in SCID mice reconstituted with human PBMCs. Moreover, FN-DC are greatly superior with respect to CD40L-matured IL-4-DC in inducing in vitro cross-priming of CD8+ T cells against viral antigens. However, the fine mechanisms underlying the features of IFN-DC remained to be determined.

In the present study we wanted to investigate whether IFN-a-driven DC differentiation could affect molecular pathways involving uptake and presentation of Ag. We reasoned that IFN-DC might be very efficient in phagocytosis of apoptotic bodies as well as other exogenous molecules promoting in turn strong T cell stimulation. Thus, we sought to explore the receptor(s) mediating this process.

We report that IFN-DC exhibit increased expression of selected SRs, among them LOX-1, and efficiently present exogenous molecules stimulating strong T cell responses.

### The Affect of IFN Exposure on DC Proteasome Subunit Composition

In this study, the special attitude of IFN-DCs to induce cross-priming of CD8+ T cells against exogenous antigens was not attributable to their increased antigen up-take and endosomal processing capacity, since no significant differences were observed in these functions between IFN-DCs and IL-4-DCs. The superior function may be explained by higher levels of costimulatory molecules and HLA class I molecules expressed by IFN-DCs as compared to mature IL-4-DCs or alternatively by the possibility that IFN-DCs are more efficient in targeting antigens onto class I processing pathway with respect to mature IL-4-DCs counterparts [13].

Priming of CD8+ T cells requires recognition through the T cell receptor of MHC class I-associated peptides. Peptides are derived from the degradation of intracellular proteins by the proteasome, a multicatalytic protease composed of three distinct catalytic b subunits called b1, b2, b5 which exhibit postacidic, tryptic-like and chymotriptic-like activity respectively. When cells are exposed to IFNg, these three catalytic subunits are substituted with new components termed LMP2 (ib1), MECL-1 (ib2)) and LMP7 (ib5)) which form the so called “immunoproteasome” [14].

Immunoproteasomes show an increased capacity to cleave after hydrophobic and basic residues, which are the most frequent residues found at the COOH terminus of the MHC class I binding peptides. It is known that during DC maturation the proteasome regulator PA28a/b and the proteins involved in antigen transport and presentation such as TAP1, TAP2 and tapasin are up-regulated [15, 16]. Moreover previous studies have reported a clear up-regulation of LMP2 and MECL-1 enzymatic activities in mature DCs [16bis].

In summary, we have shown that the use of IFN, especially type 11FN, to culture immature DC, leads to IFN-DCs that have a phenotype similar to “mature” DC's that have been cultured in the presence of IL-4, but still retain the phagocytic activity of immature DC's, thus increasing their ability to cross-prime CD8+ T cells to antigens.

The invention may include at least one of the following advantages:

1. The capability of partially mature IFN-DCs of antigen uptake and of endosomal processing is similar to that of immature IL-4-DCs.
2. In IFN-DCs the uptake of apoptotic cells is mainly mediated by LOX-1, at variance with what observed for IL-4-DCs.
3. In IFN-DCs LOX-1 mediates the cross-presentation of allogeneic apoptotic cell-derived antigens to autologous CD8+ T cells.
4. IFN-DCs exhibit an overall proteasome enzymatic activity that is higher than that exerted by proteasomes isolated from mature DCs (IL-4-DCs treated with LPS).
5. IFN-DCs are superior in cross-presenting low amounts of the soluble HCV NS3 protein to the specific CD8+ T cell clone, although all DC types tested in our studies efficiently cross-presented this viral antigen.
6. IFN-DCs are superior with respect to CD40L-matured IL-4-DCs in inducing the in vitro cross-priming of HIV-specific CD8+ T cells.
7. IFN-DCs are superior with respect to CD40L-matured IL-4-DCs in inducing in vitro cross-priming of purified CD8+ T cells in the virtual absence of helper CD4 T cells.
8. IFN-DCs pulsed with viral antigens (inactivated HIV-1) and injected into hu-PBL-SCID mice are superior with respect to CD40L-matured IL-4-DCs in inducing the in vivo cross-priming and expansion of virus-specific CD8+ T cells.
9. IFN-DCs are more potent than mature IL-4-DCs in stimulating the cytotoxic activity of CTLs specific for a sub-dominant CD8+ epitope of the EBV LMP-2 antigen.

### EXPERIMENTS

### Materials and Methods

### Cell Separation and Culture

Peripheral blood mononuclear cells were obtained from heparinized blood of healthy donors by Ficoll density gradient centrifugation (Seromed). Monocytes were isolated by immunomagnetic selection (MACS Cell Isolation Kits; Miltenyi Biotec). Positively selected CD14⁺ monocytes were plated at the concentration of 2×10⁶ cells/ml in AIM-V medium (GIBCO BRL), supplemented with 2% autologous plasma, 500 U/ml GM-CSF and either 250 U/ml IL-4 (R&D Systems) for 6 days or 10,000 U/ml natural IFN-α (Alfaferone; AlfaWasserman) for 3 days. DCs were matured by treatment with CD40L (1 μg/ml)+0.1 μg/ml enhancer (Alexis Biochimicals) for one additional day. CD40L and enhancer kit Human rhsCD40L FLAG® Set is a commercial kit from Alexis corporation. The extracellular domain of human CD40L (CD154) (aa 116-261) is fused at the N-terminus to a linker peptide (6 aa) and a FLAG®-tag. FLAG is a registered trademark of Sigma-Aldrich Co. The “Enhancer” for Ligands (Prod. No. ALX-804-034) increases the biological activity of rhsCD40L at least 1,000-fold by ligand crosslinking. Human CD8⁺ T cells were isolated by positive immunomagnetic selection (MACS Cell Isolation Kits; Miltenyi Biotec).

### Cell Lines

Lymphoblastoid cell lines (LCL) were established by in vitro infection of B lymphocytes from healthy donor typing for HLA-A2+ or HLA-A11, A28 with B95.8 strain of EBV. LCL was cultured in RPMI-1640 supplemented with 2 mM glutamine, 100 IU/ml penicillin, 100 μg/ml streptomycin, and 10% heat-inactivated fetal calf serum (HyClone, Euroclone).

The T2 TAP-deficient HLA-A2-positive cell line was cultured in IMDM (Euroclone) supplemented with 10% FCS, 10⁻⁵ M 2-ME, 1-glutamine, penicillin/streptomycin, sodium pyruvate, nonessential amino acids, and HEPES (Euroclone).

### DCs and CTLs Lines

Peripheral blood mononuclear cells (PBMC) were obtained from heparinized blood of healthy donors by Ficoll density gradient centrifugation (Seromed). DC preparations were obtained as previously described [11]. Briefly, DCs were generated from immunomagnetically purified CD14+ monocytes (MACS Monocyte Isolation Kit; Milteny Biotec), plated at the concentration of 2×10⁶ cells/ml in AIM-V medium (GIBCO BRL) and supplemented with 500 U/ml GM-CSF and either 250 U/ml IL-4 (R&D System) for 5 days or 10000 U/ml IFN-α-2b (IntronA) for 3 days. IL-4-DC were matured by treatment with 1 μg/ml of LPS (Sigma-Aldrich) for one additional day. HLA-A2-restricted EBV-specific CTL cultures reacting against the LMP2 epitope CLG, were obtained by stimulation of lymphocytes from a HLA-2-positive EBV-seropositive donor with peptide pulsed T2 cells as previously described [19]. CTL cultures were maintained in medium supplemented with 10 U/ml rIL-2 (Chiron, Milan, Italy). PHA-activated blasts were obtained by stimulation of PBLs with 1 mg/ml of purified PHA (Well-come Diagnostics, Dartford, UK) for 3 days and expanded in medium supplemented with 10 U/ml rIL-2, as described previously (Gavioli et al., 1993, J. Virology). HLA-A2-restricted EBV-specific CTL cultures reacting against the epitope LMP2₄₂₆₋₄₃₄ were obtained by stimulation of monocyte-depleted PBLs from EBV-seropositive donor RG (HLA-A2, -B8, 44) with peptide-pulsed T2 cells (Micheletti et al., 1999 Eur. J. Immunol.). The second and third stimulations were performed under the same conditions on days 7 and 14, respectively. Medium was supplemented from day 8 with 10 U/ml recombinant IL-2. The specificity of CTL was tested against a panel of EBV-positive and negative targets, including the autologous LCLs and PHA-activated blasts, allogeneic LCLs sharing HLA-A2 and HLA-A2-mismacthed LCLs.

### Immunophenotypic Analysis

Cells were washed and resuspended in PBS containing 1% human serum and incubated with a anti-CD80 (Becton Dickinson), CD40, CD86, CD83, HLA-ABC, DC-SIGN, CCR5, CXCR4 and CD4 (BD PharMingen). Cells were analyzed by flow cytometry by using a FACSort™ (Becton Dickinson) flow cytometer.

### Phagocytosis

DCs (0.5×10⁶ cells) were incubated for 60 min at 37° C. with either 50 μg/ml of dextran-FITC conjugate or 10 μg/ml of DQ-Ovalbumin (Molecular Probes). Cells were washed and resuspended in 500 μl of PBS. DCs incubated with either dextran-FITC or DQ-Ovalbumin at 4° C. were used as control. Cells were analysed by flow cytometry. To evaluate the capacity of IFN-DC and immature IL-4-DCs to internalize tumor cells, 10⁷/ml LCL cells were stained with PHK67 green (fluorescent cell linker mini kit Sigma), washed 3× in RPMI 1640 (GIBCO), resuspended in 3 ml of AIMV medium at 1.5×10⁶/ml (GIBCO), irradiated for 3′ with 400 mJ/cm² UV-B to induce apoptosis and finally incubated at 37° C. After 20 hours apoptotic cells were >70%, as evaluated by annexin-V FITC and propidium iodide (PI) staining (DB Pharmingen).

LCL cell lysate was obtained after three cycles of rapid freezing/thawing, which induce death in 95% cells, as assessed by trypan blue dye. Phagocytic activity was then evaluated by incubating DCs with apoptotic or LCL lysate (1:2 DC:tumor cell ratio) in AIMV medium at 37° C. or 4° C. (specificity control). After 4 h or alternatively an over night culture, DCs were stained with the PE-anti-CD11c mAb and phagocytic cells were identified as double-positive events.

### Peptide and Peptide Pulsing

The synthetic peptides used in this study correspond to LMP2-derived CLGGLLTMV (CLG, aa 426-434) epitope. Peptide CLG was synthesized by solid phase method and purified by HPLC to >98% purity, as previously described (Micheletti et al., 2000). Structure verification was performed by elemental and amino acid analysis and mass spectrometry. Peptide was dissolved in DMSO at 10⁻² M, kept at −20° C., and diluted in PBS before use.

For peptide pulsing, 2×10⁶ stimulator or target cells were incubated with 50 μl of peptide 10⁻⁵ M for 2 h at 37° C., washed and then added to responder cells.

### Western Blot Assay

Equal amounts of proteins were loaded on a 12% SDS-PAGE and electroblotted onto Potran nitrocellulose membranes (Scheleicher & Schuell Microscience, Keene, N.H.). Blots were probed with Abs specific for α subunits, LMP2, LMP7, MECL1, PA280α and PA28β subunits (Affinity) or with 148.3 anti TAP1 monoclonal antibody (kindly provided from Dr. Tampé Frankfurt), T2 1-435 monoclonal Ab for TAP2 (kindly provided from Dr. Van Endert Paris), or with a rabbit anti Tapasin Ab (kindly provided from Dr. Momburg Heidelberg) and developed by ECL (Amersham Biosciences, Uppsala, Sweden).

### Enzymatic Assays

To purify proteasomes, DCs were resuspended in cold buffer containing 50 mM Tris-HCl pH 7.4, 5 mM MgCl₂, 1 mM dithiothreitol (DTT), 2 mM ATP, 500 μM EDTA pH 8 and 10% glycerol. Glass beads equivalent to the volume of cellular suspension were added and cells were vortexed for 2 min at 4° C. Glass beads and cell debris were removed by centrifugation for 5 min at 1000 g followed by centrifugation at 10000 g for 20 min. Supernatants were then subjected to sequential ultracentrifugation for 1 h and then 5 h at 100000 g to obtain a 5-h pellet containing proteasomes (Gaczynska, M., et al. Nature 365, 264-267, 1993). Protein concentration was determined using the BCA (BiCinchoninc Acid) method (Pierce, Rockford, Ill.).

The 5-h pellets is resupsend in 0.5 ml activity buffer (50 mM Tris-HCl pH 7.4, 5 mM MgCl₂ and 500 μM EDTA, 1 mM dithiothreitol (DTT) and 2 mM ATP) and used to measure the protease activity. Fluorogenic substrates (100 μM) detecting chymotryptic-like (Suc-LLVY-AMC), tryptic-like (Boc-LRR-AMC) and post-acidic (Z-LLE-MCA) activities were incubated for 90 min at 37° C. with semi-purified proteasome in activity buffer in a final volume of 100 μl. The fluorescence was determined by fluorimeter (Tecan, SPECTRAFluor) with excitation at 380 nm and emission at 460 nm. Proteasome activity is expressed as arbitrary fluorescence units (A.F.U.)

### Antigen Presentation Assay of the HCV-NS3 Protein to the Specific CD8⁺ T Cell Clone

Cells of a CD8⁺ T cell clone specific for the HLA-A2 binding peptide NS31406-1415 (KLVALGINAV) of the HCV-NS3 c33c recombinant protein [39] were stimulated with the relevant peptide or with protein-loaded DC (either IFN-DCs or IL-4-DCs) in U-bottom microculture wells at 2×10⁴ DC/3×10⁴ CD8³⁰ T cell/well in 0.2 ml of RPMI 1640 medium supplemented with 10% foetal calf serum (RPMI-1640-10%). DCs loaded with either the peptide or the NS3 protein for 18 h at 37° C. were then washed with RPMI-1640-10% and added to the culture at the DC/T cell ratio of 1:1.5. After a 2-h culture, the cells were further treated with brefeldin-A (10 μg/ml, Sigma-Aldrich) at 37° C. for 18 h. Cells were washed and stained with anti-CD8 tricolor (TC) (Caltag Laboratories, Burlingame, Calif., USA) for 20 min at 4° C., fixed, permeabilized using Cytofix/Cytoperm solution (BD Pharmingen) at 4° C. for 20 minutes, rewashed with Perm Wash Buffer (BD Pharmingen), intracellularly stained with FITC-labeled anti-IFN-γ antibody (BD Pharmingen) for 30 min at 4° C. and finally subjected to flow cytometry.

### T Cell Stimulation

DCs derived from EBV-seropositive donors were loaded with autologous LCL lysates or autologous apoptotic LCL at a DC:LCL ratio 1:2 for 4 hours. Loaded DCs were seeded in replicate wells of 96-well plates at 10⁴ cells/well in 100 μl of AIM-V (GIBCO) supplemented with 5% human serum AB (HS) (EuroClone), 2 mmol/L L-glutamine, 100 IU/ml penicillin/streptomycin, 1 mM sodium pyruvate, 1 mM non-essential aminoacids, 10 mM Hepes (complete medium). Responder PBLs (10⁵ cells/well) were added in 100 μl of complete medium. On day 7, 14, 21 T cells were restimulated with lys-LCL or apo-LCL-loaded DCs generated from cryopreserved monocytes, as described for the first stimulation. Recombinant hIL-2 (Collaborative Biomedical Products, Bedford, Mass.) was added on day 3 after the first stimulation (20 U/ml), and on day 3 after each restimulation (50 U/ml). The frequencies of reactive T cells was evaluated in IFN-γ ELISPOT assays performed on day 28.

### ELISPOT Assay

IFN-γ ELISPOT assays were performed on day 28. MultiScreen-HTS plates (Millipore, Bedford, Mass.) were coated with 100 ml of 10 mg/ml of capture monoclonal antibodies (mAbs) anti-human EFN-γ (Mabtech) for 20 hours at room temperature. Plates were then washed three times with PBS and blocked with complete medium for 2 h at 37° C. DC-stimulated T cell lines (5×10³ cells/well) were incubated with autologous lys-LCL-loaded DCs as well as autologous or allogeneic LCL as APCs, at responder to stimulator ratio 1:1. After incubation at 37° C. for 20 h, plates were extensively washed with PBS-0.2% Tween 20, and incubated for 2 h at room temperature with 100 ml of 2 mg/ml biotinylated secondary mAb anti-human IFN-g (Mabtech). After extensive washing, 100 ml of streptavidine-alkaline phosphatase (ALP) conjugated (dil. 1:1000) (Mabtech) was added to the wells, and the plates were incubated for 60 min at room temperature. Colorimetric reaction was obtained using alkaline phosphatase conjugate substrate ALP (Mabtech). The number of spots was automatically determined with the use of a computer-assisted video image analyzer (Aelvis).

### Cytotoxicity Assay

CTLs responder were tested for their cytolytic activity in standard 4-h ⁵¹Cr release assay. DCs or HLA-A2 LCL pulsed or unpulsed with CLG peptide, apo-LCL-loaded DCs and apoptotic or intact HLA-A2-mismatched LCL, were labelled with 100 μCi sodium-51 chromate (PerkinElmer Life Sciences, Boston, Mass.), extensively washed, and used as target cells (10000 targets/well) at various E:T ratios, as indicated. The percentage of specific ⁵¹Cr release was calculated as follows: (mean experimental cpm-mean spontaneous cpm)/(mean maximum cpm-mean spontaneous cpm)×100%. Spontaneous release was >20% of maximum release.

### Detection of HIV-1 Infection in DC Cultures

DCs (IFN-DC and ILA-DC) were washed and infected with HIV-1 SF162 strain for 2 hours at 37° C. After extensive washing, DCs were cultured in RPMI containing 10% FCS at the concentration of 10⁶ cells/ml. Culture medium was harvested at day 3. For PCR detection of HIV-1 proviral sequences, DNA was extracted from DCs. The presence of human sequences was determined by DNA-PCR using specific primers for the HLA-DQα gene:

| | |
|---|---|
| GH26 | |
| 5′GTGCTGCAGGTGTAAACTTGTACCAG3′, | (SEQ ID NO. 1) |
| and | |
| GH27 | |
| 3′CACGGATCCGGTAGCAGCGGTAGAGTTG5′. | (SEQ ID NO. 2 |
| | in 5′-3 |
| | orientation) |

HIV-1 proviral DNA was detected by specific amplification of HIV-1 gag sequences:

| | |
|---|---|
| GAG 881 | |
| 5′GGTACATCAGGCCATATCACC3′, | (SEQ ID NO. 3) |
| and | |
| GAG 882 | |
| 3′ACCGGTCTACATAGTCTC5′. | (SEQ ID NO. 4 |
| | in 5′-3 |
| | orientation) |

The sensitivity of the assay was tested by amplifying serial dilutions of DNA prepared from 8E5 cells (which harbour one proviral copy/cell). 8E5 DNA was serially diluted into human cell DNA. Virus replication was determined after 3 days of culture by detection of p24 gag antigen in culture supernatant using a commercial ELISA kit (Dupont, Bruxelles, Belgium).

Immunization of hu-PBL-SCID Mice

CB17 scid/scid female mice (Charles River Laboratories) were used at 4 wk of age. Three or four mice for each group were injected i.p. with 30-40×10⁶ PBLs resuspended in 0.5 ml AIM-V medium. To prepare the inactivated HIV-1, different SF-162 HIV-1 stocks were inactivated by treatment for 1 h at 37° C. with 2,2′-dithiodipyridine (aldrithiol-2 [AT-2]) as described elsewhere (6). Four or seven days after reconstitution, hu-PBL-SCID mice were injected i.p. with 2.5×10⁶ autologous DCs pulsed for 2 h at 37° C. with AT-2-inactivated HIV-1 (100 ng p24). Mature DCs were loaded with antigens prior to the induction of maturation by sCD40L treatment. The vaccinated mice received one boost immunization at day 7 and were sacrificed after additional 7 days.

### ELISA for Human Immunoglobulins

Sera from control and vaccinated hu-PBL-SCID mice, collected at 7 and 14 days after the first immunization, were tested for the presence of antibodies to HIV-1 by an ELISA system for quantifying human immunoglobulins to the AVERY HIV-1 gp41 epitope, based on the use of anti-human total IgG or IgM (Cappel-Cooper Biomedical), as described in detail elsewhere [7].

Recovery of Cells from hu-PBL-SCID Mice and ELISPOT Assay

Hu-PBL-SCID mice were sacrificed 7-10 days after the last immunization. Cells were collected from the peritoneal cavity and spleen. Human cells from mouse spleens were enriched by Ficoll density gradient centrifugation and pooled (3-4 mice per group). Autologous DCs were pulsed for 2 h at 37° C. with AT-2-inactivated HIV-1 (100 ng p24), washed and used as APCs for stimulation of human cells recovered from hu-PBL-SCID mice. Control uninfected DCs were used as stimulators for the calculation of background spots. PBMC cultures treated with 2 μg/ml PHA served as positive controls. The cells were added at 10⁶ per well and incubated at 37° C. overnight in a final volume of 2 ml of AIM-V medium (GIBCO) supplemented with 2 mM L-glutamine and 2% heat-inactivated autologous plasma. After incubation with autologous DCs at a responder/stimulator ratio of 4:1, CD8⁺ T cells were positively selected by MACS Micro Beads (Miltenyi Biotec) and tested 10⁵/well in an ELISPOT assay for the production of IFN-γ (Euroclone Ltd.)[7].

### In Vitro Induction of Cross-Priming of CD8⁺ T Cells Against HIV-1 Antigens by Using Either Purified CD8⁺ T Cells or Total PBLs

CD8⁺ T cells and PBLs (4×10⁶) were stimulated with 10⁶ autologous IFN-DCs or mIL-4-DCs, pulsed with AT-2-inactivated HIV-1 (100 ng of p24) for 2 h at 37° C. In the case of ILA-DCs, cells were first loaded with antigens and subsequently induced to maturation by sCD40L treatment. CD8⁺ T cells and PBLs were restimulated 7 days later with HIV-pulsed DCs. Seven days later, the frequency of HIV-1-specific T cells was evaluated by ELISPOT assays for IFNγ (Euroclone) or granzyme-B (Becton Dickinson) according to the manufacturer's instructions. Ten-fold dilutions (from 10⁵ to 10²) of DC-stimulated CD8⁺ T cells and PBLs from primary cultures were restimulated overnight with DCs pulsed with inactivated HIV-1 (E/S ratio of 1:1), added to duplicate wells, and incubated for 18 h. Control uninfected DCs were used as stimulators/targets for the calculation of background spots to be subtracted for the evaluation of the specific number of IFN-γ or granzyme-B-spot-forming cells. PBMCs cultures treated with 2 μg/ml PHA served as positive controls. IFN-γ or granzyme-B-producing cells was evaluated by enumerating single spots using an automatic analyzer.

### Detection of Cytokine Production

Commercial ELISAs were used to quantitate in the cell culture supernatants the following cytokines: IL-6, IL-2, IL-1β, IL-12 and TNF-α (Endogen), IL-23 (Bender MedSystem), IL-7 (D.R.G.), IL-10 and IL-15 and TGF-β1 (R&D Systems), IL-18 (M.B.L.) and for measirng PGE₂ (Assay, Designs, Inc.). Assay sensitivity was as follows: IL-6 (10.24 pg/ml), IL-7 (15.6 pg/ml), IL-10 (3.6 pg/ml), IL-12 (25.6 pg/ml), IL-23 (78 pg/ml), IL-15 (3.9 pg/ml), IL-18 (25.6 pg/ml), TNF-α (15.6 pg/ml), IL-2 (38.4 pg/ml), IL-1β (10.24 pg/ml, TGFβ1 (31.2 pg/ml) and PGE₂ (39.1 pg/ml). ELISAs were performed in triplicate and laboratory standards were included on each plate.

Evaluation of IL-23 and IL-27 Subunit mRNA Expression by Real-Time RT-PCR Analysis

DCs were obtained from blood monocytes as described above and then induced to differentiate by overnight exposure to sCD40L. To measure cytokine mRNA expression, TaqMan real-time reverse transcriptase PCR (RT-PCR) analysis was used (Applied Biosystems, Foster City, Calif.). Total RNA was extracted from monocytes and DCs at different time points, and reverse transcribed. TaqMan assays were performed according to the manufacturer's instructions with an ABI 7700 thermocycler (Applied Biosystems). PCR was performed, amplifying the target cDNA (p40, and p19 transcripts for IL-23. EBI-3 and p28 for IL-27), with β-actin cDNA as an endogenous control. Data were analyzed with the PE Relative Quantification software of Applied Biosystems. At time zero, mRNA levels, normalized to β-actin, were determined for each individual cytokine chain and were expressed relative to β-actin mRNA. Specific mRNA transcript levels were expressed as fold increase over the basal condition (untreated monocytes).

### Results

IFN-DCs Highly Resemble pDCs

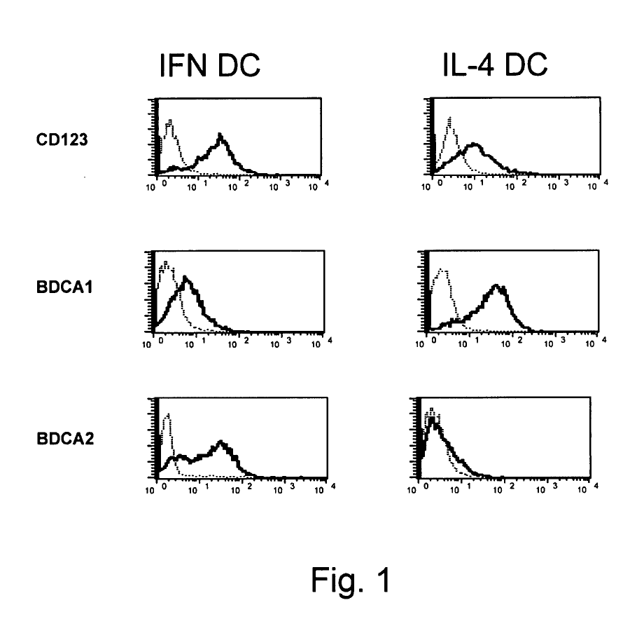

As previously described (Parlato et al., 2001, 98:3022-9), IFN-DCs expressed high levels of the lymphoid DC marker CD123 (IL-3Ra) which was poorly detected in IL-4-DCs. Notably, a remarkable percentage of IFN-DCs expressed the plasmacitoid marker BDCA2 which was undetectable in IL-4-DCs; on the contray, the IFN-DCs exhibited a marked reduction in the expression of BDCA1 myeloid marker, which was consistently expressed in IL-4-DCs (FIG. 1). These results showed that DCs generated after exposure of monocytes to type I IFN exhibited a phenotype very similar to mature DCs and in particular to CD123⁺-BDCA2⁺-plasmacitoid dendritic cells.

As expected, GM-CSF-DCs exhibited a phenotypic profile very similar to immature dendritic cells generated with GM-CSF and IL-4 (data not shown).
Comparison between IFN-DCs and IL4-DCs for Capabilities of Antigen-Uptake and Endosomal Processing

Firstly, we performed a set of experiments aimed at evaluating whether the higher capability of CD8⁺ T cell cross-priming by the IFN-DCs with respect to the IL-4-DCs [7] could be associated with an enhanced attitude of antigen uptake and endosomal processing. Antigen uptake by DCs is mediated predominantly by either mannose receptor-mediated endocytosis or macropinocytosis, which are modulated during DC differentiation. We have evaluated mannose receptor-mediated endocytosis by measuring the uptake of FITC-conjugated dextran polysaccharide, while macropinocytosis and endosomal processing capacity has been evaluated by the uptake of DQ ovalbumin, which is a self-quenched conjugate of albumin exhibiting bright green fluorescence upon endo-lysosomal protease-dependent degradation, thus permitting the evaluation of both antigen uptake and processing by live DCs. FIGS. 2A and 2B show the phenotype of the two types of DCs used in these experiments. Consistently with previously published results [6], IFN-DCs were characterized by a higher percentage of cells expressing CD40, CD80, CD86 (FIG. 2A). The up-regulation of membrane expression of these markers (FIG. 2B) was also associated with the appearance of the DC maturation marker CD83⁺. Notably, IFN-DCs nearly exhibited a two-fold increase of HLA Class-I molecule expression intensity as compared to IL-4 DCs (FIGS. 2B and 17A). As illustrated in FIG. 2C, no major difference in the dextran uptake capacity was detected between the two DC types (IFN-DCs and IL-4-DCs) (FIG. 2C). Likewise, both DC types exhibited similar FACS profile after incubation with DQ ovalbumin (FIG. 2D), suggesting that the majority of cells retained comparable phagocytic and processing activity. In particular, time-course analyses of antigen uptake and processing revealed similar kinetics for both DC types (data not shown). Thus, the finding that both DC types exhibited a similar capability of antigen uptake and processing suggested that other mechanisms were responsible for the special attitude of IFN-DCs to induce cross-priming of CD8⁺ T cells against exogenous viral antigens.

### Evaluation of the Phagocytic Activity of IFN-DCs vs Immature IL-4-DCs.

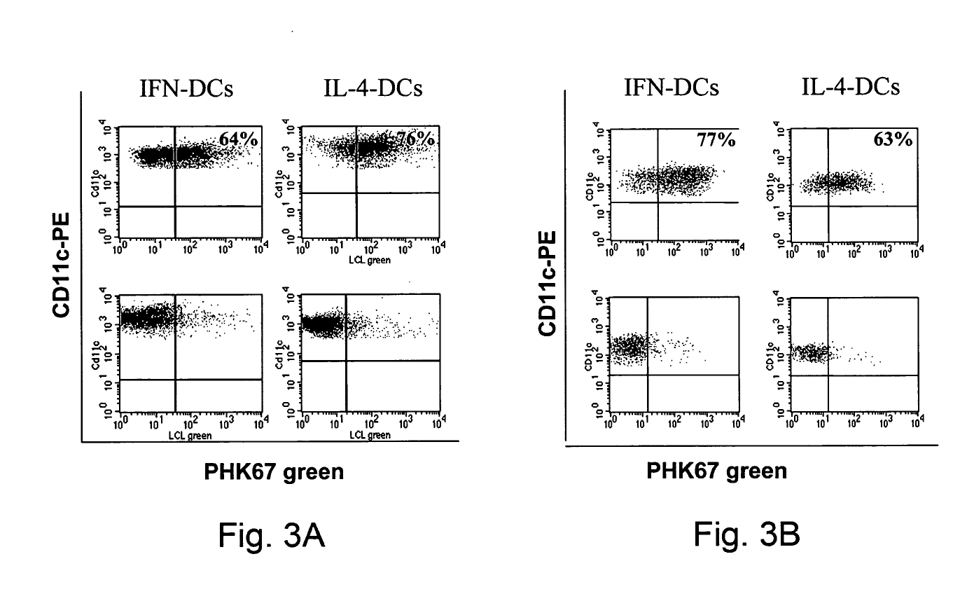

Firstly, we comparatively evaluated the ability of IFN-DCs and IL-4-DCs to phagocytose apoptotic tumor cells or tumor cell lysates. The DCs were stained with anti-CD11c antibody and co-cultured at 37° C. or 4° C. for 4 hours with PHK67 green-labelled apoptotic LCL cells or LCL cell lysates. After co-cultivation, the number of CD11c⁺-PHK67⁺ double-positive DCs was assessed by flow cytometry analysis (FIG. 3). The mean percentage (±SD) of IFN-DCs actively uptaking apoptotic cell-derived material or cell lysates calculated in several independent experiments was 64.2±5.4 (n=5) or 77.3±6.0 (n=3), respectively, and very similar to that observed in the corresponding co-cultures with IL-4-DCs, namely 76.3±4.5 and 62.6±8.6.

These results indicate that IFN-DCs, despite their partially mature phenotype, exhibit a significant phagocytic activity, similar or even superior to that of classical immature DCs.

### IFN-DCs Up-Regulate Scavenger Receptor Genes

In order to investigate the molecular mechanisms activated by IFN-alpha during the DC activation/differentiation process, we have performed global transcript analysis in IFN-DCs compared to monocytes treated with GM-CSF alone and to DCs generated with GM-CSF and IL-4 by using Affymetrix platform. Thus, we selected four different donors whose dendritic cells generated in vitro with IFN-alpha, IL-4 or GM-CSF displayed immunophenotypic features typical of mature or immature dendritic cells, respectively. Total RNA extracts were obtained from non-adherent cells and amplified antisense RNA (aRNA) was hybridized to Affymetrix HG U133A oligonucleotide arrays covering 14,500 well-characterized human genes. Significant Analysis of Microarray (SAM) method was used to select the genes significantly modulated by IFN- and IL-4-treatments with respect to the common control (GM-CSF). We obtained two lists of genes corresponding to a global list of 807 genes, significant for at least one of the two treatments

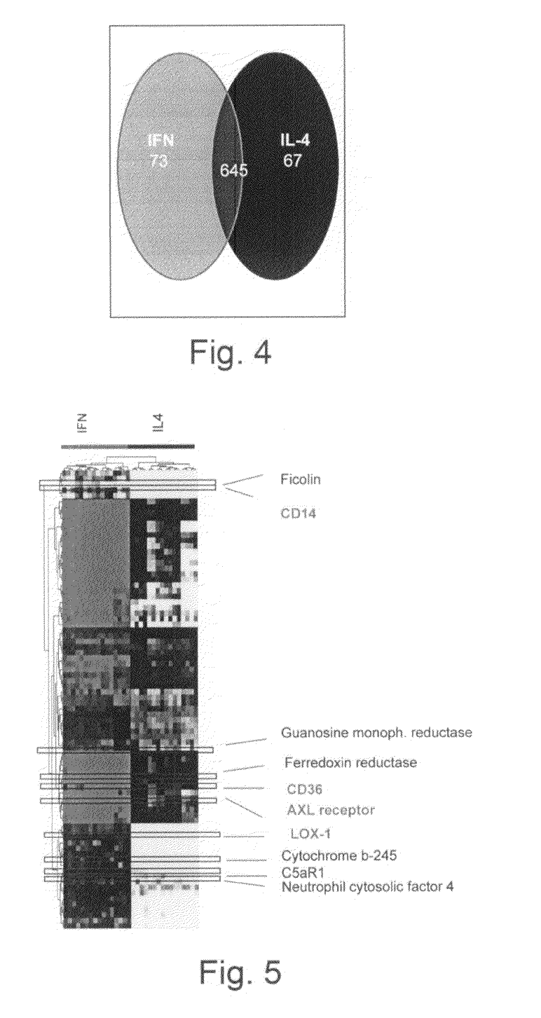

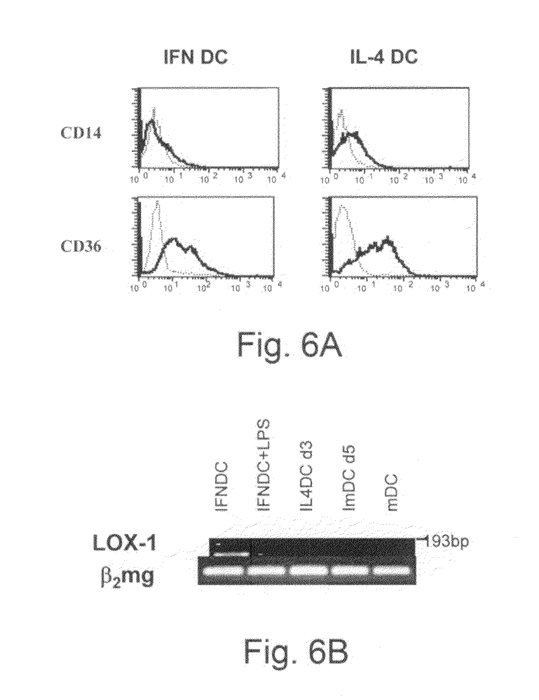

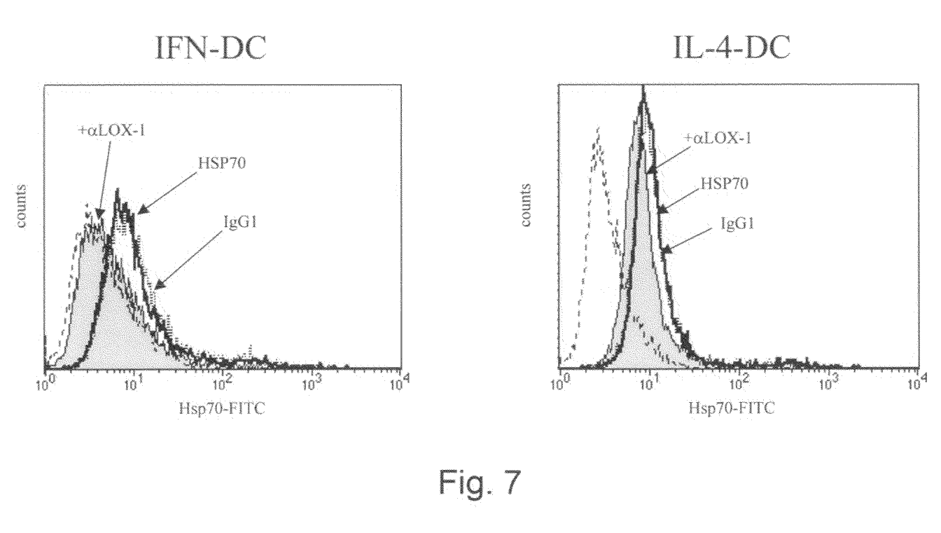

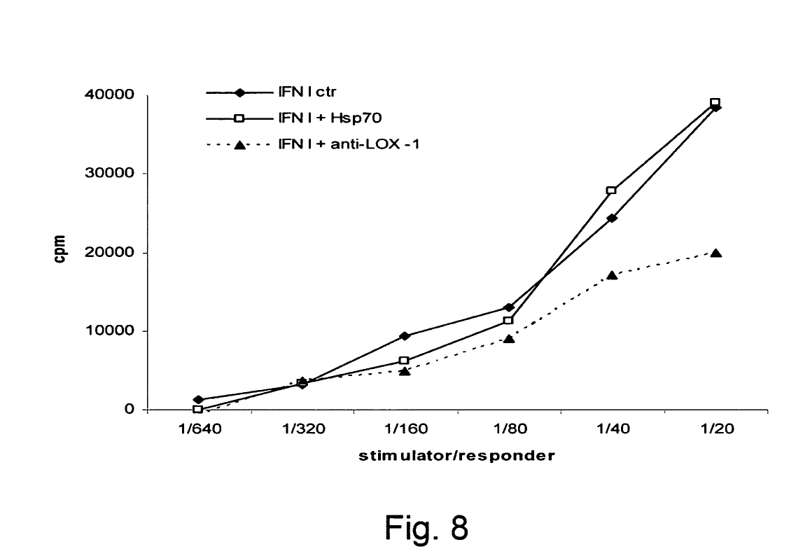

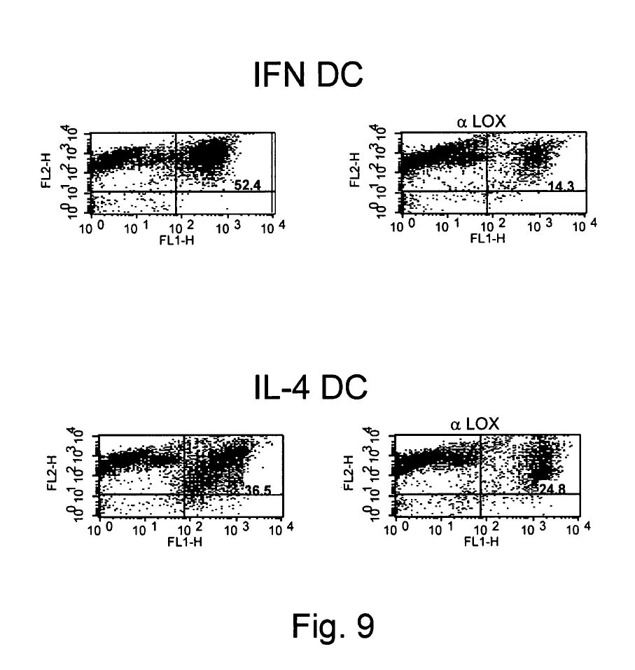

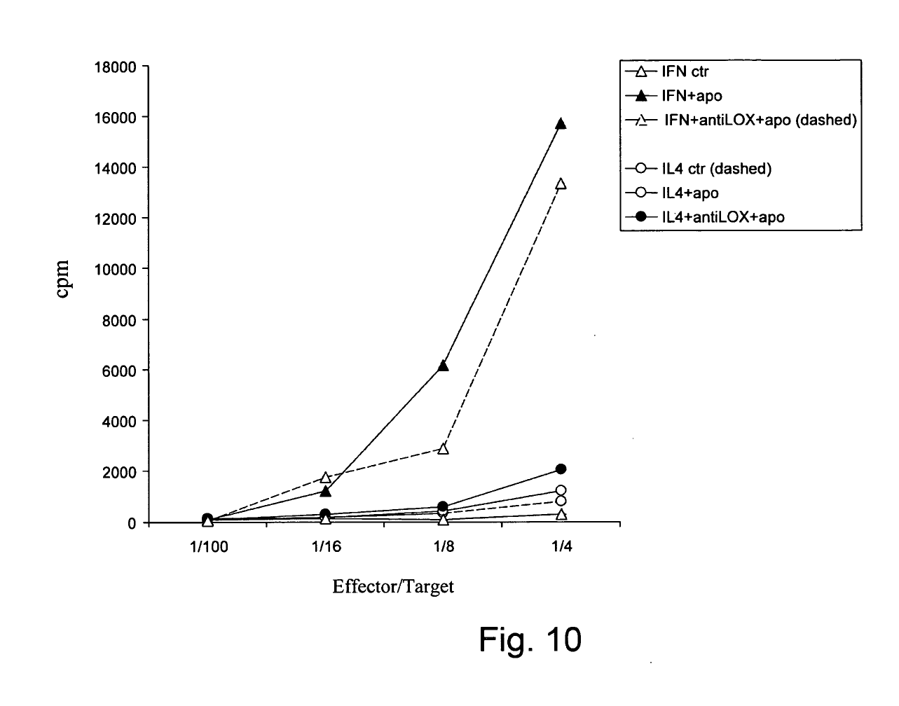

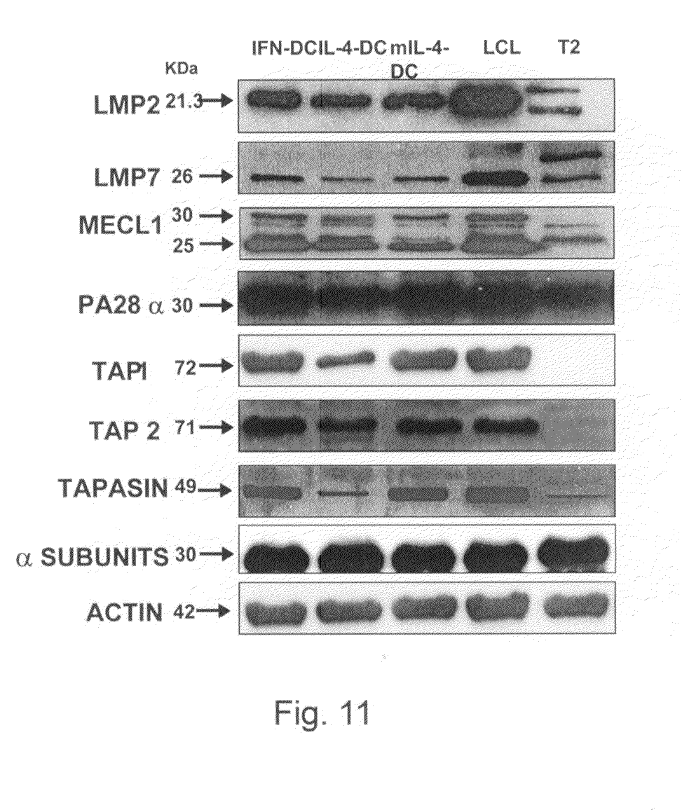

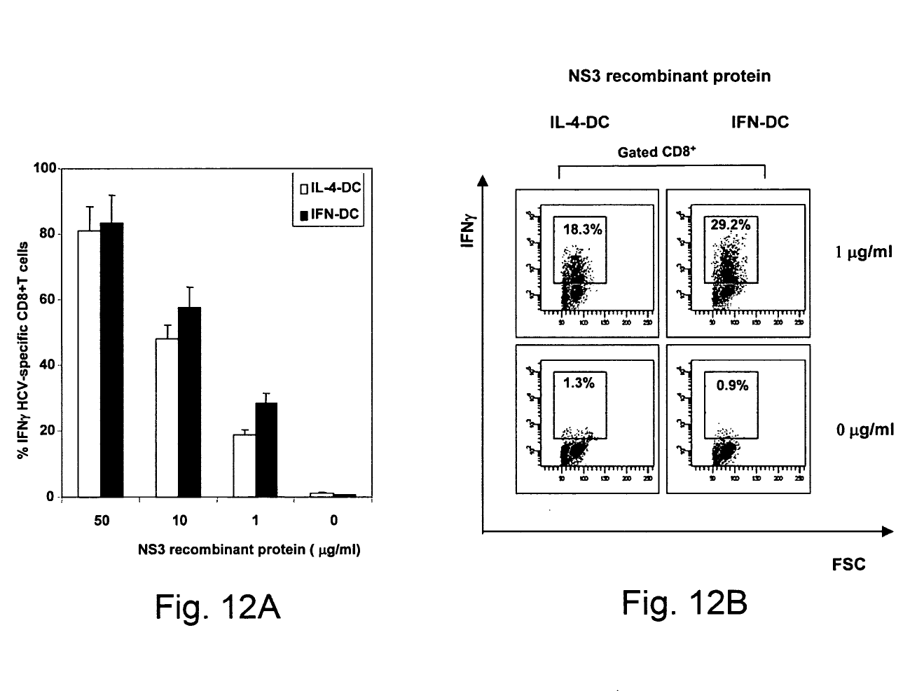

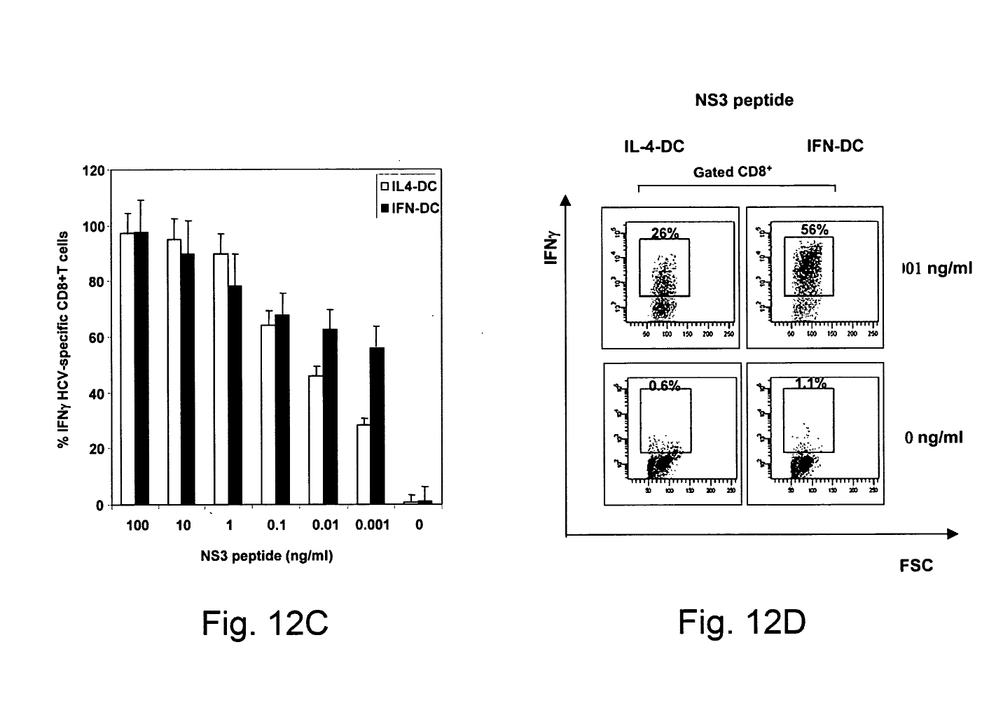

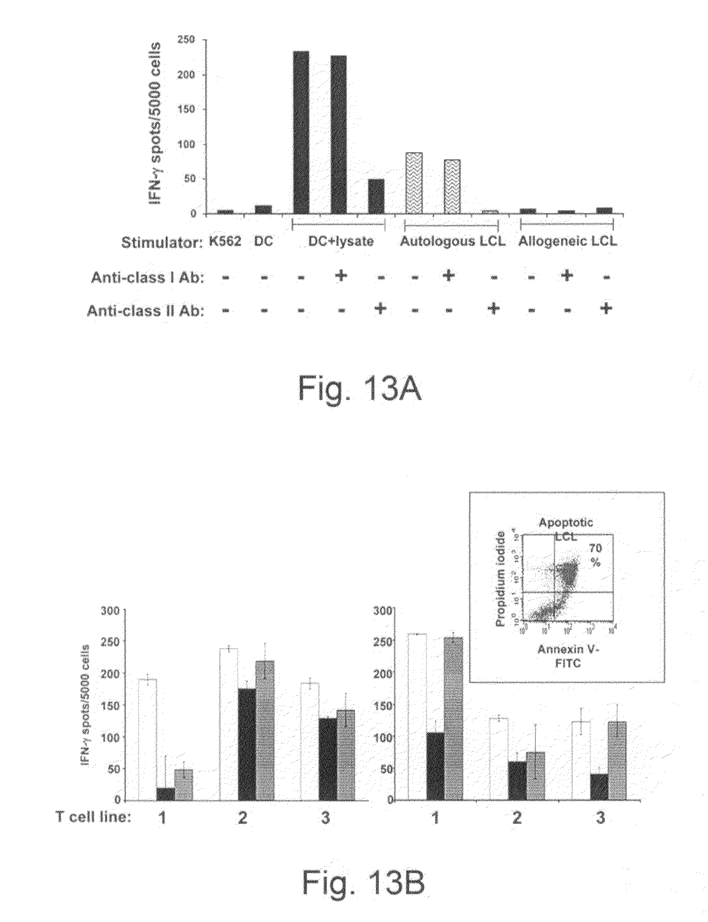

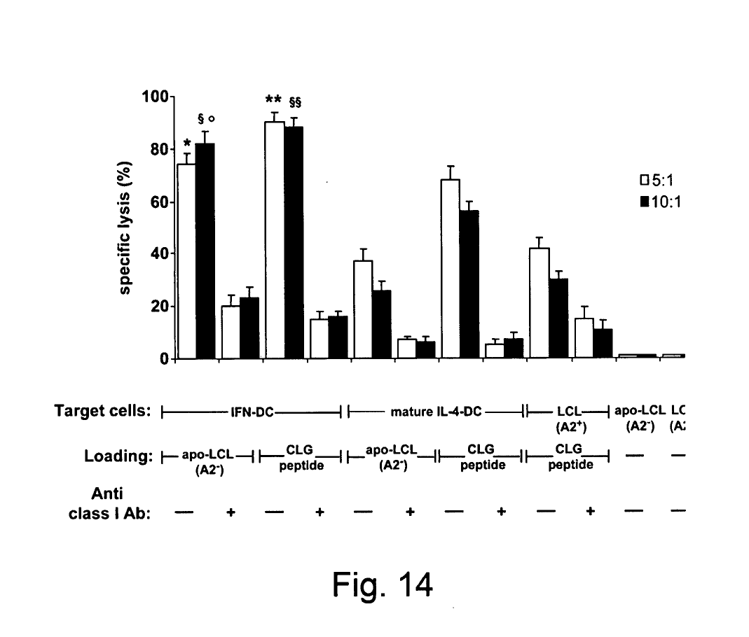

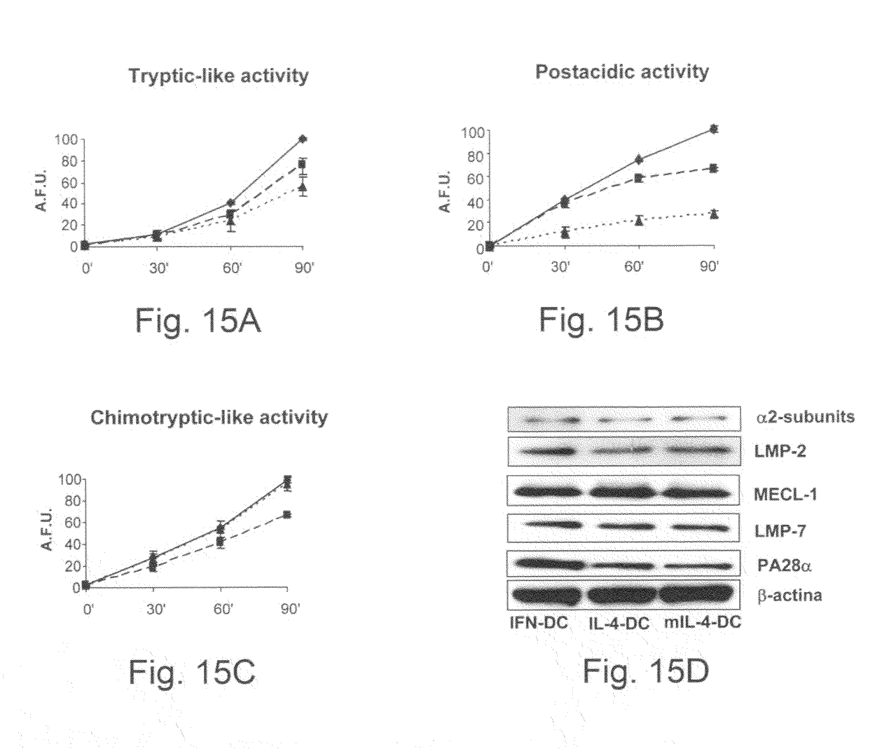

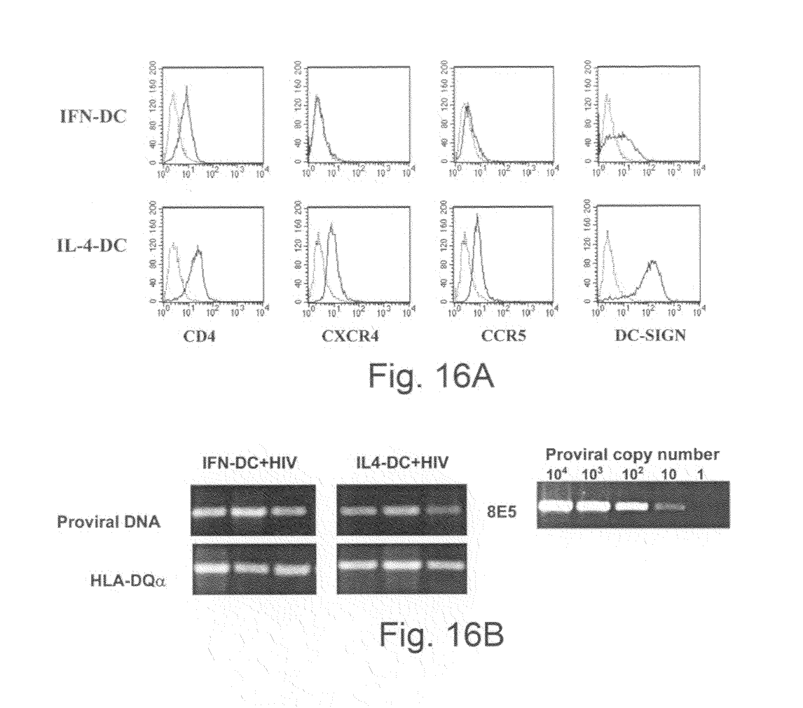

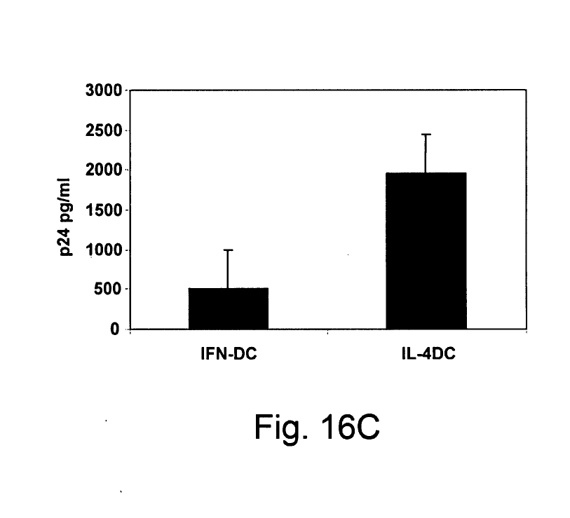

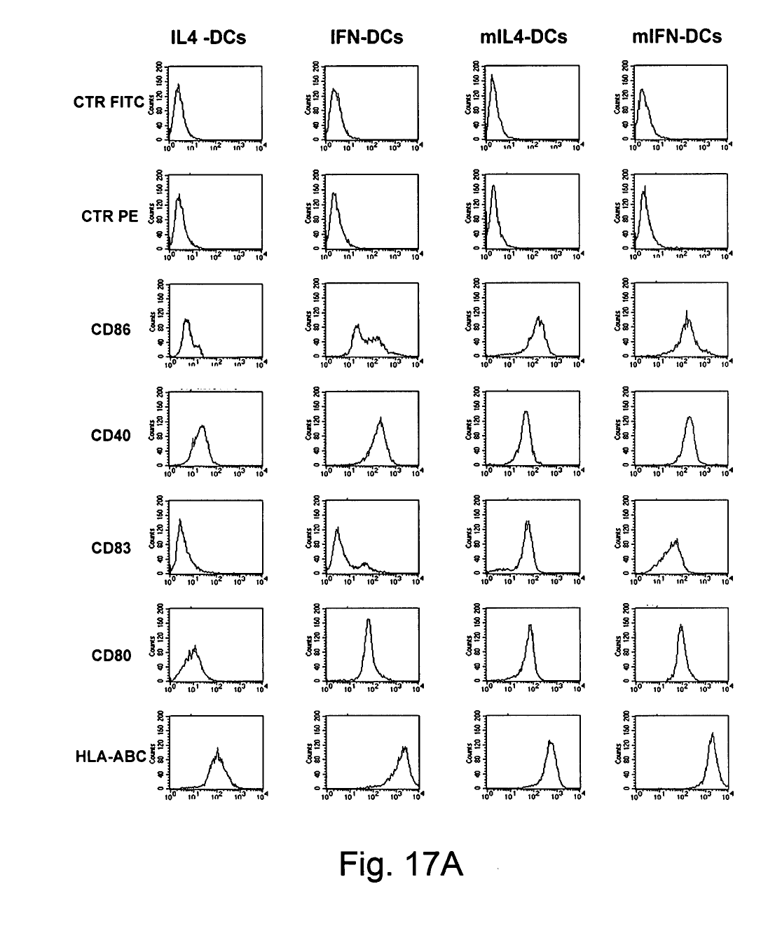

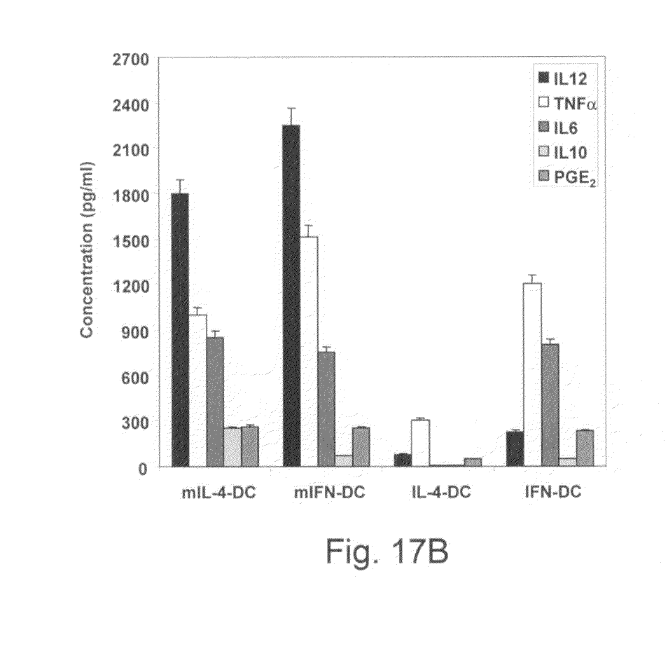

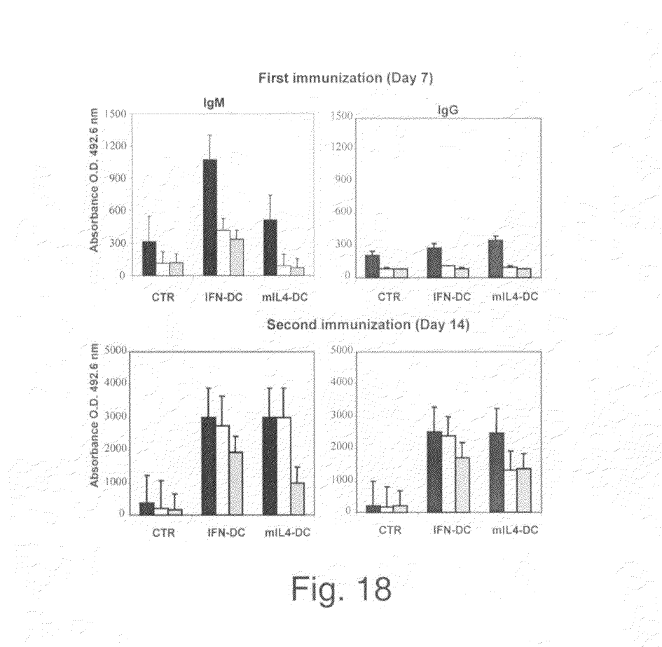

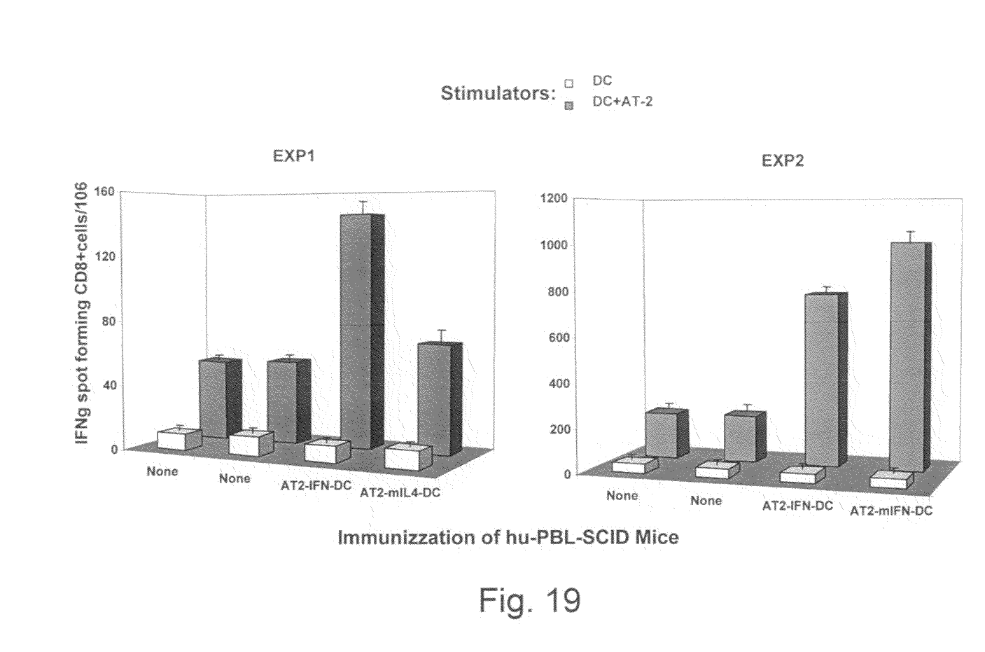

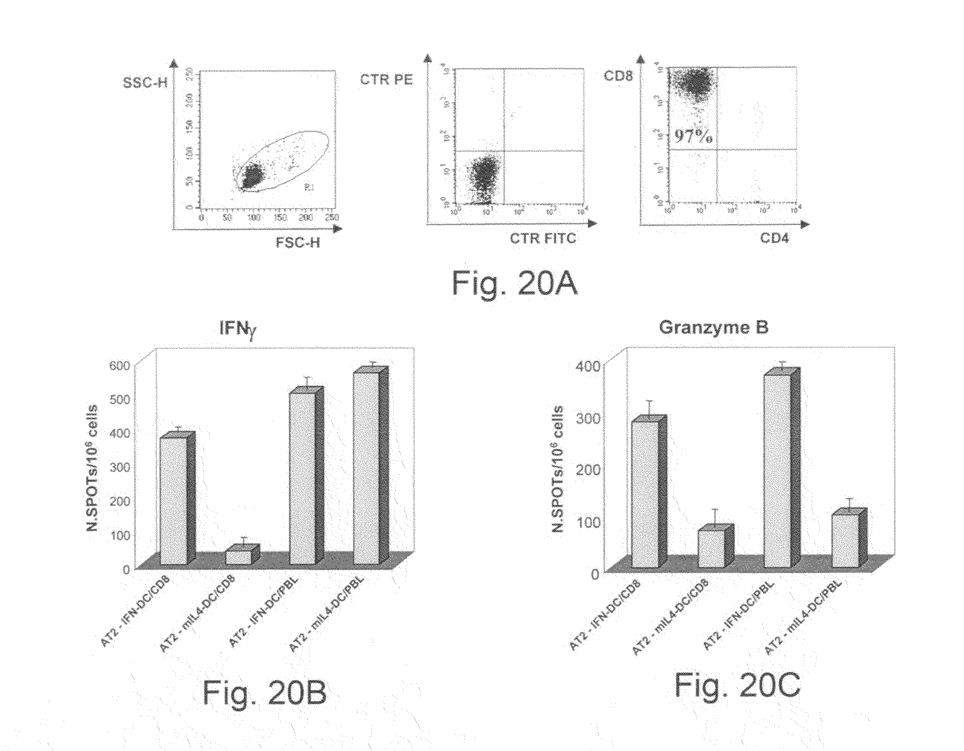

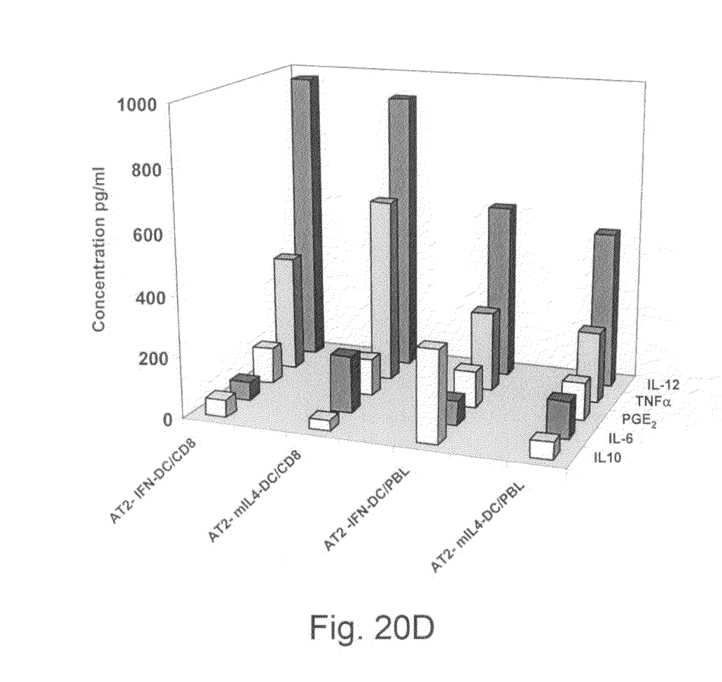

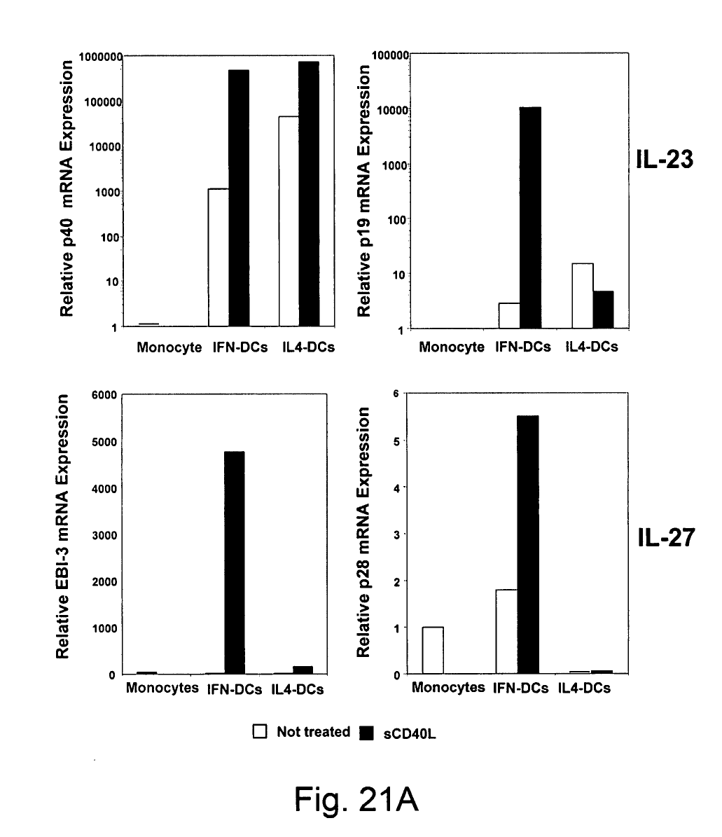

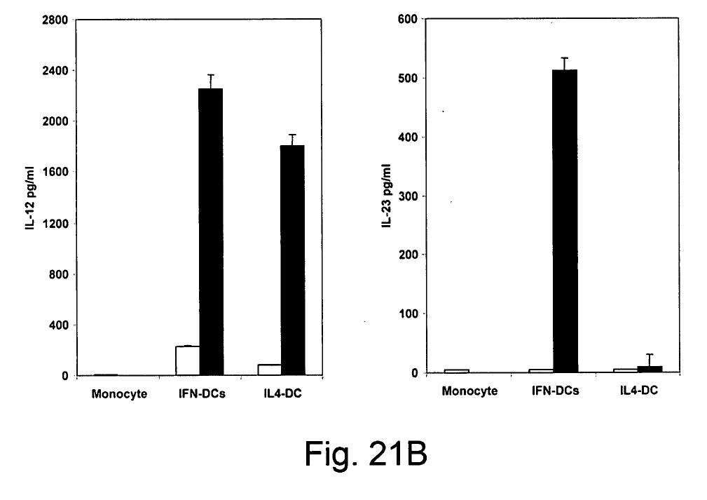

Thus, a second round of SAM analysis was performed to select genes differentially modulated by the two treatments. As summarized by the Venn diagram (FIG. 4), type I IFN treatment significantly up-regulated 73 genes with respect to IL-4 treatment, whereas 67 genes were up-regulated in the IL-4- compared to IFN-treatment; 645 genes were not differentially modulated by the two treatment.

GO categories analysis (http://david.niaid.nih.gov/david/ease.htm) showed substantial differences in terms of over-expressed gene families modulated by the two treatment. As shown in Table 1, the addition of IFN-alpha to human monocytes induced an over-expression of gene categories involved in immunological pathways such as innate immune response, inflammatory response, chemotaxis, signal transduction, cytokine and chemokine activity, antigen processing and presentation. On the contrary, the IL-4 treatment mainly induced genes related to metabolic pathways. Notably, the IFN-induced gene families had a very high statistical significance (Benjamini adj. fact. from 2e-019) in comparison to those modulated by IL-4 (Benjamini adj. fact. from 8e-003).

To further characterize the gene expression signature of IFN-DCs and ILA-DCs, a hierarchical algorithm was applied to each set of genes belonging to Venn diagram groups (data not shown). Hierarchical cluster analysis confirmed the strong differences in gene profiles between the two DC population. As summarized in Table 2, the IFN-DCs showed, as expected, a strong induction of the best characterized IFN-inducible genes (2′-5′-OAS, IFIT2, IFIT4, ISG20, IFITM2, viperin, IF127) and of transcription factor genes belonging to the IRF family (IRF2 and IRF7). Moreover, IFN-DCs showed an up-regulation of mRNA encoding proteins involved in inflammatory response (S100A8, S100A9, MyDD88), chemotaxis (CCL8, CX3CR1, EP10, CXCL3, CXCL2), apoptosis and cytotoxicity (Fas, TRAIL, caspase 1), antigen processing, transport and presentation (LMP2, TAP1, MHC class I), as compared to IL-4-DCs. Interestingly, hierarchical cluster of 73 genes modulated by IFN-treatment contained the mRNA encoding some proteins involved in endocytic and phagocytic processes (FIG. 5).

In particular, IFN exposure induced a strong up-regulation of CD14, LOX-1, CD36 and AXL genes belonging to the Scavenger Receptor family. SRs are cell-surface glycoproteins involved in uptake and clearance of modified host molecules, exogenous components and apoptotic cells. SRs are expressed by certain endothelial cells but also by myeloid cells (macrophages and dendritic cells) playing an important role in innate immune response.

Capture of exogenous antigens and apoptotic cell-derived antigens is a key step for DCs to initiate an immune response. In particular, LOX-1, CD14, AXL and CD36 receptors are able to recognize a wide range of negatively charged macromolecules, including oxidized low-density lipoproteins, apoptotic cells and components of pathogenic microorganisms (Yamada Y, Peiser). The up-regulation of the expression of these molecules following IFN-treatment was confirmed by FACS analysis and semi-quantitative RT-PCR analysis (FIG. 6). Notably, results from RT-PCR analysis showed that LOX-1 was exclusively expressed by IFN-DCs whereas it was completely lost following maturation stimulus (LPS). Moreover, was only weakly detectable in IL-4-DCs and in classical immature (ImDC) and mature (mDC) monocyte-derived dendritic cells (FIG. 6B).

TABLE 1
Over-expressed gene categories in IFN- and IL-4-DCs

| Treatment | GO system | GO category | Benjamini |
|---|---|---|---|
| IFN | Biological process | immune response | 2.4e−019 |
| | ″ | defense response | 4e−019 |
| | ″ | response to external stimuli | 4.7e−016 |
| | ″ | organismal physiological | 5.3e−014 |
| | | process | |
| | ″ | response to pathogen/ | 1.6e−007 |
| | | parasite | |
| | ″ | inflammatory response | 5.6e−006 |
| | ″ | innate immune response | 7.2e−006 |
| | ″ | response to stress | 1.7e−004 |
| | ″ | chemotaxis | 2.5e−003 |
| | Molecular Function | signal transducer activity | 3e−002 |
| | ″ | cytokine activity | 3e−002 |
| | Biol. Proc. | phisiological process | 3e−002 |
| | Mol. Func. | chemokine activity | 4.7e−002 |
| | Biol. Proc. | apoptosis | 4.7e−002 |
| | Mol. Func. | transmembrane receptor | 1e−001 |
| | | activity | |
| | Biol. Proc. | antigen presentation | 1.3e−001 |
| | ″ | antigen processing | 1.4e−001 |
| IL-4 | Molecular function | oxidoreductase activity | 7.9e−003 |
| | Biol. Process | lipid metabolism | 3.9e−002 |
| | ″ | fatty acid metabolism | 1.6e−001 |
| | Mol. Func | RNA binding | 1e+000 |
| | Biol. Proc. | biosynthesis | 1e+000 |
| | ″ | macromolecule biosynthesis | 1e+000 |
| | ″ | carboxylic acid metabolism | 1e+000 |
| | ″ | organic acid metabolism | 1e+000 |
| | Cell. Component | membrane | 1e+000 |
| | Mol. Func. | catalytic activity | 1e+000 |

NOTE:
Over-expressed GO categories in IFN-DC and IL-4-DC are shown with decreasing significance (as indicated by Benjamini value).

TABLE 2
Selected genes higher expressed in IFN-DCs in comparison to IL-4-DCs

IFN-RELATED GENES
IFN alpha-inducible protein 27
IFN-induced transmembrane
protein 1
Viperin
2′-5′-OAS-like
IFN alpha inducible protein
(clone IFI-6-16)
Guanylate binding protein,
IFN-inducible (67 kD)
IFN responsive protein 28 kD
ISG20
IFN-induced protein with
tetratricopeptide repeats 2
IFN-induced protein with
tetratricopeptide repeats 4
IFN-induced transmembrane
protein 2
2′-5′-OAS 3 and 2′-5′-OAS 2
CHEMOTAXIS
CCL-8 (MCP-2)
CX3CR1 (fractalkine receptor)
CXCL-10 (IP-10)
CXCL-3 (GRO gamma)
CXCL-2 (GRO beta)
C5aR1
APOPTOSIS/CYTOTOXICITY
Fas
TRAIL
Caspase 1
IL-1beta
DUSP6
DEAD/H box polypeptide
BIRC 3
Synuclein, alpha
SIGNAL TRANSDUCTION
IRF2
IRF7
MARCKS-like protein (MLP)
PILRa
CLECSF5(c-type lectin, similar to
DAP12-associating lectin)
INFLAMMATORY RESPONSE
Calgranulin A (S100A8)
Calgranulin B (S100A9) and MyDD88
CYTOKINE RECEPTORS
IL-7R
UBIQUITIN CYCLE
USP18
Ubiquitin-conjugating enzime E2L&
HERC6
Ag PROCESSING/TRANSPORT
MHC class I
Galectin-3
TAP1
LMP2
PHAGOCYTOSIS/ENDOCYTOSIS
AXL receptor
LOX-1
CD14
CD36
Ficolin
Cytochrome b245
Ferredoxin reductase
Guanosine monophosphate reductase
Neutrophil cytosolic factor 4
C5aR1

### LOX-1-Mediated Hsp70 Binding Stimulates IFN-DC Function

LOX-1 (lectin-like oxidized low-density lipoprotein receptor-1) was first identified as an endothelial cell-specific scavenger receptor which can bind, internalize and degrade oxidized low-density lipoprotein (oxLDL) (Oka 1997). Recently, Delneste et al. showed that human macrophages and peripheral blood myeloid dendritic cells constitutively express LOX-1, whereas it was undetectable on T cells and mature DCs. Moreover, in the same work they have reported that LOX-1 is one of the scavenger receptors involved in Hsp70 binding on human DCs and that it is involved, as Hsp70-receptor, in cell-mediated antigen cross-presentation and activation of innate immune response (Delneste, 2002). It's well established that Heat shock proteins (HSP) exert immunoregulatory effects by carrying, for example, both chaperoned pro-peptide and danger signal to dendritic cells. The cross-presentation of Hsp-chaperoned peptides occur through specific endocytic receptors present on DCs, whereas the interaction of Hsp with TLR members resulted in pro-inflammatory cytokines production and up-regulation of co-stimulatory molecules conferring an adjuvant, peptide-independent activity to Hsp (Massa et al, 2005; Asea A. et al. 2002).

We have shown that IFN-DCs are dendritic cells endowed with potent functional activities both in vitro and in vivo, particularly efficient in inducing a Th-1 immune response and cross-priming of CD8+ T cells against exogenous antigens (Santini, JEM, 2000; and the present application). In the light of these data, we focused our further attention on the role of LOX-1 in the adjuvant activity exerted by Hsp70 toward IFN-DCs.

First, we showed that both IFN-DCs and IL-4-DCs can bind recombinant human Hsp70-FITC (34.7% and 42.3%, respectively), but this binding was exclusively prevented in IFN-DCs by using a neutralizing anti-LOX-1 mAb (clone 23C11), obtaining about 48% inhibition. On the contrary, the anti-LOX-1 mAb did not affect the Hsp70 recognition by IL-4-DCs (FIG. 7). The partial inhibition of Hsp70 binding by the anti-LOX-1 mAb suggests that other molecules belonging to SR family are involved in the Hsp70 binding to IFN-DCs, in according to the hypothesis formulated by Delneste and coll. (Delneste, 2002) with regard to Hsp70 binding to human immature DCs.

Further, to investigate the functional role of LOX-1 as Hsp-receptor in IFN-DCs we have performed an MLR assay evaluating the allostimulatory activity of IFN-DCs exposed to exogenous Hsp70 in the presence or in absence of neutralizing anti-LOX-1 mAb. As shown in FIG. 8, a 20 min. Hsp70 pre-treatment of IFN-DCs induced a strong capability to stimulate the proliferation of allogeneic lymphocytes, in a similar manner with respect to the untreated IFN-DCs. The presence of a neutralizing anti-LOX-1 mAb blocked the stimulation capability of Hsp70 in IFN-DCs, confirming the functional involvement of LOX-1 in the Hsp70-mediated activation of IFN-DCs. On the contrary, the pre-treatment of immature IL-4-DCs with Hsp70 and with the anti-LOX-1 mAb did not affect their allostimulatory activity (data not shown).

### IFN-DC Highly Take-Up and Present Apoptotic-Bodies via LOX-1

SRs are also involved in uptake and clearance of apoptotic cells, playing an important role in innate immune response (Peiser). We have shown that IFN-DCs exhibit a high capability to take up, process and cross-present tumor-associated antigens derived from apoptotic tumor cells to autologous CD8+ T lymphocytes (unpublished observations). Based on this we have also focused on LOX-1, known to be involved in calcium-dependent recognition of phosphatidylserine (PS) and apoptotic cells (Murphy J E). To this purpose, we have investigated the role of LOX-1 in IFN-DC capability of capturing, processing and cross-presenting Ags derived from apoptotic cells.

We have performed phagocytosis FACS assay in the presence or in absence of a neutralizing anti-LOX-1 mAb (clone 23C11) (FIG. 9). IFN- and IL-4-DCs showed the same capability to uptake apoptotic gamma-irradiated allogeneic PBLs. Of interest, the anti-LOX-mAb only inhibited the phagocytosis of apoptotic cells by IFN-DCs (about 22% of inhibition), whereas the pre-treatment of IL-4-DCs with the same neutralizing mAb did not affect their capability to phagocyte apoptotic debris.

Several works have demonstrated that the IFN-DCs are potent APCs able to induce a strong Th-1 immune response and CD8+ T cell responses against defined antigens in different models (Santini, 2000; Santodonato 2003; Gabriele 2004; Blanco 2001; Mothy 2003; Carbonneil 2003; and present work).

We correlated the phagocytic capability of IFN-DCs with their ability to cross-present antigenic material derived from early apoptotic cells by LOX-1 involvement.

To this purpose, IFN- and IL-4-DCs were co-cultured with apoptotic allogeneic PBLs in the presence or absence of anti-LOX-1 mAb and then co-cultured with autologous purified CD8+ T cells at different stimulator/responder ratios. FIG. 10 shows that only IFN-DCs were able to cross-prime autologous CD8⁺ T cells, but the cross-presentation of apoptotic bodies-derived antigens was notably inhibited when phagocytosis of apoptotic cells was carried out in the presence of neutralizing anti-LOX-1 mAb.

It is generally assumed that only mature DCs can induce cross-priming of CD8+ T cells against exogenous antigens. This is further confirmed by the observation that IL4-DCs failed in inducing CD8+ T cell proliferative response to apoptotic bodies-derived antigens (FIG. 10).

### Expression of Immunoproteasome Subunits and TAPs Proteins in IFN-DCs as Compared to IL4-DCs.

We then evaluated the expression level of the immunoproteasome subunits (PA28α/ [symbol] , LMP2, LMP7, MECL-1) and of proteins involved in the intracellular pathway of MHC class I antigen-processing machinery (TAP1, TAP2 and tapasin), in IFN-DCs as compared to immature IL-4-DCs or IL-4-DCs exposed for 48 hours to LPS. All DC types expressed an equal amount of total proteasomes, as demonstrated by Western Blot analysis with antibodies specific for the constitutive α2 proteasome subunits (FIG. 11). Interestingly, IFN-DCs exhibited levels of expression of the PA28α regulator subunit as well as of the catalytic β subunits LMP2, LMP7 and MECL-1 superior to those expressed by immature IL-4-DCs and similar to those induced by maturation of IL-4-DCs with LPS. Similarly, also the expression of TAP1, TAP2 and tapasin proteins was up-regulated in IFN-DCs as compared to IL-4-DCs and comparable to that observed in mature DCs (FIG. 11).

### Cross-Presentation of Exogenous Soluble Antigens to CD8⁺ Cells by Monocyte-Derived DCs

It was surmised that IFN-DCs are endowed with an enhanced capability to cross-present viral antigens to CD8⁺ T cells, compared to IL-4-DCs. This hypothesis has been addressed by experiments aimed at evaluating the efficiency of both DC types loaded with a reference viral soluble protein to activate a CD8⁺ T cell clone specific for the viral antigen. In particular, in a series of 3 experiments with DCs from different donors, we have studied the presentation of exogenous HCV NS3 protein to a HLA-A2-restricted NS3₍₁₄₀₆₋₁₄₁₅₎-specific CD8⁺ T cell clone [19].

The response was evaluated by intracellular staining of IFN-γ-producing cells followed by flow cytometry. First, by using cells from the same donors, we performed a series of cross-presentation assays using the same CD8⁺ T cell clone and the same DCs loaded with the whole recombinant NS3 protein. As shown in FIG. 12A, IFN-DCs showed a cross-presentation capability comparable to that of the IL-4-DCs when loaded with protein concentrations of 50 and 10 μg/ml. However, the IFN-DCs proved to be superior in cross-presenting antigen at lower protein concentration (FIGS. 12A and 12B). Consistently, when the DCs were loaded with high concentrations of the corresponding NS3 peptide, the activation of the CD8⁺ T cell clone by either IFN-DCs or IL-4-DCs proved to be similar (FIG. 12C). However, at very low peptide concentration (0.01 or 0.001 ng/ml), clone activation by IFN-DCs was significantly more efficient (FIG. 12C). This was also supported by the dot-plot analysis of IFN-γ production by the specific T cell clone when stimulated with DCs loaded with 0.001 ng/ml of the NS3 peptide, which clearly showed that IFN-DCs were associated with higher number of IFNγ-producing cells and a stronger florescence intensity (FIG. 12D).

### Evaluation of the Ability of IFN-DCs to Cross-Present LCL-Associated Antigens to CD8+ T Lymphocytes in a Totally Autologous Setting.

In order to assess the efficiency of IFN-DCs as compared to IL-4-DCs in the cross-presentation of tumor-associated antigens, we chose a completely autologous model system in which DCs from EBV-positive donors were loaded with apoptotic cells (apo-LCL) or cell lysates (lys-LCL) derived from autologous LCL, and then used as APCs for the stimulation of autologous PBMCs. After four in vitro stimulations, the frequencies of T cells specifically secreting IFN-γ in response to apo-LCL- or lys-LCL-loaded DCs versus autologous LCL were assessed by ELISPOT assays.

In the case of the PBMC cultures stimulated with lys-LCL-loaded DCs, 4 independent T cell lines specifically recognizing intact autologous LCL as well as autologous immature IL-4-DCs previously exposed to lys-LCL could be expanded when IFN-DCs were used as APCs, whereas a single specific T cell line was obtained after stimulation with lys-LCL-loaded IL-4 DCs (data not shown). For all these T cell lines, the number of cells specifically secreting IFN-γ was significantly reduced in the presence of an anti-MHC class II antibody, whereas no changes were caused by the addition of an anti-MHC class I antibody as compared to control cultures (data not shown).

The preferential expansion of a class II-restricted T cell response specific for autologous LCL after PBMC stimulation with lys-LCL-loaded IFN-DCs was confirmed by a detailed analysis in ELISPOT assays of the specificity of the T cell line exhibiting the highest frequency of IFN-γ-secreting cells (FIG. 13A). The number of IFN-γ spots observed after stimulation of this T cell line with lys-LCL-loaded IL-4-DCs was drastically reduced in the presence of an anti-MHC class II antibody, whereas it was not affected by addition of an anti-MHC class I antibody. A significant number of IFN-γ spots were observed after stimulation of the T cell line with autologous LCL, whereas no IFN-γ secretion was detected in response to allogeneic LCL, unloaded DCs or NK-sensitive K562 cells. Similar to the results obtained with lys-LCL-loaded DCs, the autologous LCL-specific response was virtually abolished by addition of an anti-MHC class II antibody whereas it was not inhibited by an anti-MHC class I antibody (FIG. 13A).

Overall, these results indicated that IFN-DCs loaded with a lysate of autologous LCL can efficiently expand a class II-restricted T cell response specific for autologous LCL, i.e. CD4⁺ T cells directed against EBV antigens.

We then evaluated the entity and specificity of the response elicited after repeated in vitro stimulation of PBMC with autologous IFN-DCs or immature IL-4DCs loaded with apo-LCL. Three independent T lymphocyte cell lines were obtained after PBMC stimulation with either DC type, with the T cell lines expanded after stimulation with apo-LCL-loaded IFN-DCs containing similar or slightly higher frequencies of T lymphocytes reactive against autologous LCL as compared to the T cell lines obtained after stimulation with apo-LCL-loaded IL-4-DCs (FIG. 13B). Similar results were obtained when purified CD8⁺ T cells were repeatedly stimulated in vitro with IFN-DCs or IL-4-DCs loaded with apo-LCL (data not shown).

In all cases, the addition of an anti-MHC class I antibody during the ELISPOT assay reduced appreciably the number of IFN-γ spots observed after stimulation of the T cell lines with autologous LCL as compared to control wells (FIG. 13B). On the contrary, no significant inhibition of autologous LCL-stimulated IFN-γ spot formation was measured in the presence of an anti-MHC class II antibody, except for one T cell line expanded after stimulation with apo-LCL-loaded IFN-DCs (T cell line indicated as 1 in the left graph of FIG. 13B).

Collectively, these observations indicated that IFN-DCs loaded with autologous apoptotic LCL could quite efficiently expand a class I-restricted T cell response specific for autologous LCL, therefore demonstrating the ability of IFN-DCs to cross-present EBV-derived TAAs to CD8⁺ T lymphocytes.

Evaluation of the Ability of IFN-DCs versus Mature IL-4-DCs to Cross-Present a Subdominant Epitope of EBV LMP-2.

We then evaluated the ability of IFN-DCs differentiated from HLA-A*0201 donors and loaded with HLA-A-mismatched apo-LCL cells to cross-present the subdominant HLA-A*0201-restricted CLG epitope of the LMP-2 EBV protein to HLA-A*0201 CD8⁺ CTLs specific for this epitope. To this end, the CLG-specific CTLs were tested in standard ⁵¹Cr release assays for their cytotoxic activity against IFN-DCs and LPS-treated IL-4-DCs both loaded with HLA-A11, A28 apo-LCL or pulsed with the CLG peptide, as well as against CLG peptide-pulsed HLA-A*0201 or HLA-A-mismatched LCL cells. When apo-LCL-loaded IFN-DCs were used as target cells of the CLG epitope-specific CTLs, a considerably higher level of specific lysis was obtained (70-80%) as compared to that reached against mature IL-4-DC counterparts (approximately 30-40%) (FIG. 14).

A much smaller difference, if any, in the extent of specific lysis was observed when the CLG-specific CTL were challenged with IFN-DCs vs mature IL-4-DCs both pulsed with the CLG epitope peptide (FIG. 14). As expected, the CLG-specific CTLs efficiently killed autologous LCL pulsed with the CLG peptide, whereas no cytotoxic activity was exerted when the allogeneic LCL used as antigen source served as target cells, either intact or apoptotic (FIG. 14). Interestingly, the level of CLG-specific CTL-mediated lysis against apo-LCL-loaded IFN-DCs was significantly superior to that exerted against CLG peptide-pulsed HLA-A*0201 LCL. In contrast, the CLG-specific CTLs killed apo-LCL-loaded mature IL-4-DCs and peptide-pulsed HLA-A*0201 LCL at a similar extent (FIG. 14).

Altogether, these observations indicated that IFN-DCs were more efficient as compared to mature IL-4-DCs in stimulating the effector function of CTLs upon cross-presentation of the specific epitope.

In order to investigate whether this functional property of IFN-DCs could be attributed to quantitative and/or qualitative characteristics of the proteasome activity, we comparatively analyzed the cleavage specificity of equal amounts of proteasomes semi-purified from IFN-DCs, immature IL-4-DCs, and IL-4-DCs treated with LPS for 20 hours (in our experimental setting, 20 hours represented the time period intervening between the addition of LPS to IL-4-DCs co-cultured with apo-LCL and the mixing of the apo-LCL-loaded DCs with the CLG-specific CTLs).

As shown in FIG. 15, both the tryptic-like (panel A) and postacidic-like (panel B) activities were augmented in proteasomes obtained from IFN-DCs as compared to both immature and LPS-treated IL-4-DCs, while the chymotryptic-like activity (panel C) was similar in IFN-DCs and LPS-treated IL-4-DCs and augmented with respect to that measured in proteasomes from immature IL-4-DCs.

These observations demonstrated that proteasomes from IFN-DCs exhibited an overall proteolytic activity higher than that exerted by proteasomes isolated from immature or LPS-treated IL-4-DCs.

The expression levels of the immunoproteasome subunits was also evaluated in total cell lysates prepared from the same DC samples used for the analysis of the enzymatic activity. The Western blotting analysis using Abs specific for the constitutive α2-subunits of proteasome, for the catalitic β subunits of immunoproteasome (LMP2, LMP7 and MECL-1) and for PA28α regulator, revealed no difference in the expression levels of the α2-subunits, suggesting that the three different DC types expressed similar amounts of total proteasomes (FIG. 15, panel D). As for the immunoproteasome subunits, all DC types expressed similar amounts of LMP7 and MECL-1 subunits, whereas IFN-DCs showed a clear up-regulation of LMP2 and PA28α subunits as compared to immature or LPS-treated IL-4-DCs. The observed increase in tryptic-like and postacidic-like activities was not in agreement with the pattern of expression of the catalytic subunits.

However, it should be noted that IFN-DCs express higher amounts of PA28α, a proteasome activator that strongly increases the proteolytic activity of proteasomes [20].

Comparison of 3-Day IFN-DCs versus CD40L-Activated IL-4-DCs for their Capability to Induce Humoral Response and Cross-Priming in hu-PBL-SCID Mice.

In a previous study based on the use of DCs pulsed with inactivated HIV-1 as antigen model, we had shown that virus-pulsed IFN-DCs were superior with respect to immature IL-4-DCs in inducing a potentially protective humoral and cellular immune response against HIV antigens when tested in hu-PBL-SCID mice [7]. However, it remained to be evaluated whether IFN-DCs could compare favorably with reference mature DCs (mIL-4-DCs), as those obtained after in vitro maturation of IL-4-DCs by exposure to CD40L. Before addressing this issue, it was also important to evaluate whether the IFN-DCs and IL-4-DCs could exhibit any differential property in interacting with HIV-1. In our previous study [7], the virus inactivation was achieved by using aldrithiol-2 (AT-2), which selectively disrupts the p7 nucleocapsid (NC) protein, thus resulting in inactivation without affecting the conformation and fusogenic activity of the gp120.

We have now analyzed the two DC types by flow cytometry for the expression of selected membrane molecules involved in viral entry. The phenotypic analysis showed lower levels of expression of membrane CD4, CXCR4, CCR5 and DC-SIGN in IFN-DCs as compared to IL-4-DCs (FIG. 16A), consistent with results from other groups [12, 13]. Similar proviral load was detected in both IFN-DCs and IL-4-DCs previously exposed to HIV (FIG. 16B). IL-4-DCs proved to be capable of releasing higher amounts of HIV with respect to the IFN-DCs (FIG. 16C). On the whole, these results suggested that the superior capability of the HIV-pulsed IFN-DCs to induce a human humoral and cellular immune response in hu-PBL-SCID mice was not due to an enhanced susceptibility of these DCs to virus entry and infection.

FIG. 17 illustrates the phenotype (FIG. 17A) and cytokine secretion patterns (FIG. 17B), before and after CD40L stimulation, of the DCs types utilized in the subsequent studies. As expected, only a small fraction of the IFN-DCs expressed the CD83 maturation marker, while the large majority of both mIL-4-DCs and mIFN-DCs were CD83⁺ (FIG. 17A). Both mIFN-DCs and mIL-4-DCs expressed comparable levels of the costimulatory molecules CD80 and CD86, higher than the corresponding immature DCs. As illustrated in FIG. 17B, IFN-DCs secreted higher amounts of TNF-α, PGE₂ and IL-6 than IL-4-DCs. Interestingly, after CD40L-induced maturation, the levels of the secreted IL-12 and TNF-αwere higher for IFN-DCs than for IL-4-DCs. In contrast, no or very low levels of secretion of IL-15, IL-18, IL-10, IL-7, TGF-β1 and IL-2 were detected in the different DC cultures (data not shown).

The immune priming activity of IFN-DCs and mIL-4-DCs pulsed with AT-2-HIV-1 was tested in hu-PBL-SCID mice, by measuring their in vivo capability to induce the generation of human antibodies and, more importantly, of CD8⁺ T cells against HIV-1 antigens. FIG. 18 shows the antibody response to HIV-1 gp41 immunodominant peptides obtained in hu-PBL-SCID mice immunized with either IFN-DCs or mIL-4-DCs loaded with AT-2-inactivated HIV-1.

At 1 week after primary and boost immunization, comparable levels of anti-HIV antibodies were detected in mouse sera, indicating that both DC types exhibited similar efficacy in the elicitation of a human antibody response. When CD40L-treated IFN-DCs (mIFN-DC) were compared with IFN-DCs, no major difference in the antibody production was observed (data not shown), suggesting that the subsequent maturation step did not result in any significant enhancement of the DC functional activity.

Interestingly, however, IFN-DCs were more efficient than mIL-4-DCs in inducing the generation of HIV-1-specific CD8⁺ T cells in the immunized hu-PBL-SCID mice, as revealed by IFN-γ ELISPOT assay (FIG. 19, Exp. 1). Notably, treatment of IFN-DCs with sCD40L did not significantly enhance the generation of HIV-specific CD8⁺ T cells (FIG. 19, Exp. 2), suggesting that IFN-DCs are fully committed to the efficient cross-priming of CD8⁺ T cells without the requirement of additional maturation steps provided by CD4⁺ T cells.

Efficient CD4⁺ T Cell-Independent Generation of Effector CD8⁺ T Cells against HIV-Antigens by IFN-DCs In Vitro.

The in vivo studies illustrated above suggested that IFN-DCs are especially effective in inducing the cross-priming of virus specific CD8⁺ T cells in vivo. Thus, we have performed in vitro experiments to characterize the capability of IFN-DCs of inducing antigen-specific effector CD8⁺ T cells against exogenous HIV antigens in the presence or absence of CD4⁺T cell help.

In particular, we compared the in vitro cross-priming of highly purified CD8⁺ T cells using the two types of AT-2-HIV-1-pulsed DCs: IFN-DCs and mIL-4-DCs. Positively selected CD8⁺ T cells represented >97% of the cell population as assessed by flow cytometry (FIG. 20A). IFN-DCs were far superior in the induction of specific CD8⁺ T cell response in absence of CD4⁺ T cell help as evaluated by both ELISPOT enumeration of IFN-γ and granzyme-B-releasing cells after restimulation with HIV-1 antigens (FIGS. 20B and 20C). Comparable numbers of IFN-γ-producing T cells were detected when the total PBLs, instead of purified CD8⁺ T cells, were co-cultured with either IFN-DCs or mIL-4-DCs (FIG. 20B). The generation of granzyme-B releasing cells was more efficiently induced by IFN-DCs both in the presence and absence of CD4⁺ T cells (FIG. 20C). FIG. 20D illustrates the production of IL-6, IL-10, IL-12, TNF-α and PGE₂ in supernatants from the last DC restimulation. Of interest, high levels of IL-12 were detected in supernatants from co-cultures of purified CD8⁺ T cells with antigen-pulsed-IFN-DCs, suggesting that IFN-DCs had acquired the full capacity to release this cytokine during co-culture. However, the differential capability of the two DC populations to induce a CD8⁺ specific T cell response (FIG. 20B and FIG. 20C) did not correlate with major differences in the pattern of cytokine production (FIG. 20D).

IFN-DCs Exhibit a High Capability to Express the IL-12 Family Cytokines IL-23 and IL-27 upon sCD40L-Induced Maturation

The results reported above showed that IFN-DCs were capable of efficiently generating an effective CD8⁺ T cell response, including the production of high levels of IFN-γ.

It was reasonable to suppose that these special property of IFN-DCs could be due to their capability to express certain cytokines involved in the amplification of the action of IL-12 and in the generation and expansion of a cytotoxic CD8⁺ T cells. In this regard, Th1 and CTL responses have been demonstrated to be promoted by the IL-12 family cytokines IL-23 and IL-27 [20-22].

Thus, we have measured the mRNA levels of IL-23 p19/p40 and IL-27 EBI-3/p28 subunits in the two types immature DCs and their corresponding mature counterparts. As shown in FIG. 21A, p40 subunit mRNA, which is shared by IL-12 and IL-23 heterodimers, was up-regulated in both IL-4-DCs and IFN-DCs at comparable levels upon maturation, while the p19 subunit was specifically up-regulated in IFN-DCs more than 1,000-fold, as confirmed by the higher levels of secreted IL-23 detected in supernatants from matured IFN-DC by ELISA (FIG. 21B). Likewise, the IL-27 EBI-3/p28 subunit mRNA levels proved to be strongly up-regulated in the IFN-DCs.

Thus, these results suggested that IFN-DCs exhibited a greater attitude to produce IL-12 family cytokines capable of supporting IL-12 activity and promoting T cell IFN-γ production.

All references cited herein are hereby incorporated by reference.

### REFERENCES

### Except for Proteasome and LOX-1 Work—See Below

1. Banchereau, J. and Steinman, R. M., Dendritic cells and the control of immunity. Nature 1998. 392: 245-252.
2. Banchereau, J., Briere, F., Caux, C., Davoust, J., Lebecque, S., Liu, Y. J., Pulendran, B. et al., Immunobiology of dendritic cells. Annu. Rev. Immunol. 2000. 18: 767-811.
3. Schuler, G., Schuler-Thurner, B. and Steinman, R. M., The use of dendritic cells in cancer immunotherapy. Curr. Opin. Immunol. 2003. 15: 138-147.
4. Kaiser, A., Bercovici, N., Abastado, J. P. and Nardin A., Naive CD8+ T cell recruitment and proliferation are dependent on stage of dendritic cell maturation. Eur. J. Immunol. 2003. 33: 162-171.
5. Sallusto, F. and Lanzavecchia, A., Efficient presentation of soluble antigen by cultured human dendritic cells is maintained by granulocyte/macrophage colony-stimulating factor plus interleukin 4 and downregulated by tumor necrosis factor alpha. J. Exp. Med. 1994. 179: 1109-1118.
6. Santini, S. M., Lapenta, C., Logozzi, M., Parlato, S., Spada, M., Di Pucchio, T. and Belardelli, F., Type I interferon as a powerful adjuvant for monocyte-derived dendritic cell development and activity in vitro and in Hu-PBL-SCID mice. J. Exp. Med. 2000. 191: 1777-1788.
7. Lapenta, C., Santini, S. M., Logozzi, M., Spada, M., Andreotti, M., Di Pucchio, T., Parlato, S. et al., Potent immune response against HIV-1 and protection from virus challenge in hu-PBL-SCID mice immunized with inactivated-virus-pulsed dendritic cells generated in the presence of IFN-α. J. Exp. Med. 2003. 198: 361-367.
8. Santodonato, L., D'Agostino, G., Nisini, R., Mariotti, S., Monque, D. M., Spada, M., Lattanzi, L. et al., Monocyte-derived dendritic cells generated after a short-term culture with IFN-α and granulocyte-macrophage colony-stimulating factor stimulate a potent Epstein-Barr virus-specific CD8⁺ T cell response. J. Immunol. 2003. 170: 5195-5202.
9. Gabriele, L., Borghi, P., Rozera, C., Sestili, P., Andreotti, M., Guarini, A., Montefusco, E. et al., IFN-α promotes the rapid differentiation of monocytes from patients with chronic myeloid leukemia into activated dendritic cells tuned to undergo full maturation after LPS treatment. Blood 2004. 103: 980-987.
10. Buelens, C., Bartholome, E. J., Amraoui, Z., Boutriaux, M., Salmon, I., Thielemans, K., Willems, F. et al., Interleukin-3 and interferon beta cooperate to induce differentiation of monocytes into dendritic cells with potent helper T-cell stimulatory properties. Blood 2002. 99: 993-998.
11. Blanco, P., Palucka, A. K., Gill, M., Pascual, V. and Banchereau, J., Induction of dendritic cell differentiation by IFN-α in systemic Lupus Erythematosus. Science 2001. 294: 1540-1543.
12. Mohty, M., Vialle-Castellano, A., Nunes, J. A., Isnardon, D., Olive, D. and Gaugler, B., IFN-alpha skews monocyte differentiation into Toll-like receptor 7-expressing dendritic cells with potent functional activities. J. Immunol. 2003. 171: 3385-3393.
13. Carbonneil, C., Aouba, A., Burgard, M., Cardinaud, S., Rouzioux, C., Langlade-Demoyen, P. and Weiss L., Dendritic cells generated in the presence of granulocyte-macrophage colony-stimulating factor and IFN-alpha are potent inducers of HIV-specific CD8 T cells. AIDS 2003. 17: 1731-1740.
14. Ridge, J. P., Di Rosa, F. and Matzinger, P., A conditioned dendritic cell can be a temporal bridge between a CD4⁺ T-helper and T-killer cell. Nature 1998. 393: 474-478.
15. Schoenberger, S. P., Toes, R. E., van der Voort, E. I., Offring a, R. and Melief, C. J., T-cell help for cytotoxic T lymphocytes is mediated by CD40-CD40L interactions. Nature 1998. 393: 480-483.
16. Bennett, S. R., Carbone, F. R., Karamalis, F., Flavell, R. A., Miller, J. F. and Heath, W. R., Help for cytotoxic-T-cell responses is mediated by CD40 signalling. Nature 1998. 393: 478-480.
17. Fujii, S., Liu, K., Smith, C., Bonito, A. J. and Steinman, R. M., The linkage of innate to adaptive immunity via maturing dendritic cells in vivo requires CD40 ligation in addition to antigen presentation and CD80/86 costimulation. J. Exp. Med. 2004. 199: 1607-1618.
18. O'Sullivan, B. J. and Thomas, R., CD40 ligation conditions dendritic cell antigen-presenting function through sustained activation of NF-kappaB. J. Immunol. 2002. 168: 5491-5498.
19. Accapezzato, D., Visco, V., Francavilla, V., Molette, C., Donato, T., Paroli, M., Mondelli, M. U. et al., Chloroquine enhances human CD8+ T cell responses against soluble antigens in vivo. J Exp Med. 2005. 202: 817-28.
20. Trinchieri, G., Pflanz, S. and Kastelein, R. A., The IL-12 family of heterodimeric cytokines: new players in the regulation of T cell responses. Immunity 2003. 19: 641-644.
21. Trinchieri, G. Interleukin-12 and the regulation of innate resistance and adaptive immunity. Nat. Rev. Imnunol. 2003. 3: 133-146.
22. Matsui, M., Moriya, O., Belladonna, M. L., Kamiya, S., Lemonnier, F. A., Yoshimoto, T. and Akatsuka, T., Adjuvant activities of novel cytokines, interleukin-23 (IL-23) and IL-27, for induction of hepatitis C virus-specific cytotoxic T lymphocytes in HLA-A*0201 transgenic mice. J. Virol. 2004. 78: 9093-9104.
23. O'Sullivan, B. J. and Thomas, R., CD40 and dendritic cell function. Crit. Rev. Immunol. 2003. 23: 83-107.
24. Valenzuela, J., Schmidt, C. and Mescher, M., The roles of IL-12 in providing a third signal for clonal expansion of naïve CD8 T cells. J. Immunol. 2002. 169: 6842-6849.
25. Kleen, T. O., Asaad, R., Landry, S. J., Boehm, B. O. and Tary-Lehmann, M., Tc effector diversity shows dissociated expression of granzyme B and interferon-gamma in HIV infection. AIDS 2004. 1: 383-392.
26. Yang, O. O., Will we be able to ‘spot’ an effective HIV-1 vaccine? Trends Immunol. 2003. 24: 67-72.
27. Cho, H. J., Hayashi, T., Datta, S. K., Takabayashi, K., Van Uden, J. H., Horner, A., Corr, M. and Raz, E., IFN-alpha beta promote priming of antigen-specific CD8+ and CD4+T lymphocytes by immunostimulatory DNA-based vaccines. J. Immunol. 2002. 168: 4907-4913.
28. Belardelli, F. and Ferrantini, M., Cytokines as a link between innate and adaptive antitumor immunity. Trends Immunol. 2002. 23: 201-208.
29. Ha, S. J., Kim, D. J., Baek, K. H., Yun, Y. D. and Sung, Y. C., IL-23 induces stronger sustained CTL and Th1 immune responses than IL-12 in hepatitis C virus envelope protein 2 DNA immunization. J. Immunol. 2004. 172: 525-531.
30. Belladonna, M. L., Renauld, J. C., Bianchi, R., Vacca, C., Fallarono, F., Orabona, C., Fioretti, M. C., et al., IL-23 and IL-12 have overlapping, but distinct, effects on murine dendritic cells. J. Immunol. 2002. 168: 5448-5454.
31. Lucas, S., Ghilardi, N., Li, J. and de Sauvage, F. J., IL-27 regulates IL-12 responsiveness of naive CD4+ T cells through Stat1-dependent and -independent mechanisms. Proc. Natl. Acad. Sci. USA 2003. 100: 15047-15052.
32. Hibbert, L., Pflanz, S., De Waal Malefyt, R. and Kastelein, R. A., IL-27 and IFN-alpha signal via Stat1 and Stat3 and induce T-Bet and IL-12Rbeta2 in naive T cells. J. Interferon Cytokine Res. 2003. 23: 513-522.
33. Le Bon, A. and Tough, D. F., Links between innate and adaptive immunity via type I interferon. Curr. Opin. Immunol. 2002. 14: 432-436.
34. Le Bon, A., Etchart, N., Rossmann, C., Ashton, M., Hou, S., Gewert, D., Borrow, P. et al., Cross-priming of CD8+ T cells stimulated by virus-induced type I interferon. Nat. Immunol. 2003. 4: 1009-1015.
35. Beignon, A. S., Skoberne, M. and Bhardwaj, N., Type I interferons promotes cross-priming: more functions for old cytokines. Nat. Immunol. 2003. 4: 939-941.
36. Figdor, C. G., de Vries, I. J., Lesterhuis, W. J. and Melief, C. J., Dendritic cell immunotherapy: mapping the way. Nat. Med. 2004. 10: 475-480.
37. O'Neill, D. W., Adams, S. and Bhardwaj, N., Manipulating dendritic cell biology for the active immunotherapy of cancer. Blood 2004. 104: 2235-2246.
38. Lu, W., Arraes, L. C., Ferreira, W. T. and Andrieu, J. M., Therapeutic dendritic-cell vaccine for chronic HIV-1 infection. Nat. Med. 2004. 10: 1359-1365.
39. Barnaba, V., Franco, A., Alberti, A., Benvenuto, R. and Balsano, F., Selective killing of hepatitis B envelope antigen-specific B cells by class I-restricted, exogenous antigen-specific T lymphocytes. Nature 1990. 345: 258-260.

### REFERENCES

### For IFN-DC and LOX-1 Work

Yamada Y, Doi T, Hamakubo T, Kodama T. Scavenger receptor family proteins: roles for atherosclerosis, host defence and disorders of the central nervous system. CMLS 54:628, 1998.
Peiser L, Mukhopadhyay S, Gordon S. Scavenger receptors in innate immunity. Curr. Opin. Immunol. 14:123, 2002.
Oka K, Sawamura T, Kikuta K, Itokawa S, Kume N, Kita T, Masaki T. Lectin-like oxidized low-density lipoprotein receptor 1 mediates phagocytosis of aged/apoptotic cells in endothelial cells. PNAS 95:9535, 1998.
Murphy J E, Tacon D, Tedbury P R, Hadden J M, Knowling S, Sawamura T, Peckham M, Phillips S, Walker J H, Ponnambalam S. LOX-1 scavenger receptor mediates calcium-dependent recognition of phosphatidylserine and apoptotic cells. Biochem. J. 393:107, 2006.
Delneste Y, Magistrelli G, Gauchat J-F, Haeuw J-F, Aubry J-P, Nakamura K, Kawakami-Honda N, Goetsch L, Sawamura T, Bonnefoy J-Y, Jeannin P. Involvement of LOX-1 in dendritic cell-mediated antigen cross-presentation. Immunity 17:353, 2002.
Massa C, Melani C, Colombo M P. Chaperon and adjuvant activity of hsp70: different natural killer requirement for cross-priming of chaperoned and bystander antigens. Cancer Res. 65:7942, 2005.
Asea A, Rehli M, Kabingu E, Boch J A, Barè O, Auron P E, Stevenson M A, Calderwood S K. Novel signal transduction pathway utilized by extracellular HSP70. J. Biol. Chem. 277: 15028, 2002.

### REFERENCES

### For IFN-DC and Proteasome Work

[1] Beignon, A. S., Skoberne, M., Bhardwaj, N., Type I interferons promote cross-priming: more functions for old cytokines. Nature Immunol. 2003. 10: 939-941.
[2] Le Bon, A., Durand, V., Kamphuis, E., Thompson, C., Bulfone-Paus, S., Rossmann, C., Kalinke U. and Tough, D. F., Direct stimulation of T cells by type I IFN enhances the CD8+ T cell response during cross-priming. J. Immunol. 2006. 176: 4682-4689.
[3] Le Bon, A. and Tough, D. F., Links between innate and adaptive immunity via type I interferon. Curr. Opin. Immunol. 2002. 14: 432-436
[4] Blankenstein, T., and Shuler, T., Cross-priming versus cross-tolerance: are two signals enough? Trends in Immunol. 2002. 23 (4): 171-173.
[5] Ridge, J. P., Di Rosa, F., & Matzinger, P. A., A conditioned dendritic cells can be a temporal bridge between a CD4+T-helper and a T-killer cell. Nature. 1998. 393: 474-478.
[6] Belz, G. T., Behrens, G. M., Smith, C. M., Miller, J. F., Jones, C., Lejon, K., Fathman, C. G., et al., The CD8a⁺ dendritic cells is responsible for inducing peripheral self-tolerance to tissue-associated antigens. J. Exp. Med. 2002. 196: 1099-1104.
[7] Schuler, G., Schuler-Thurner, B. and Steinman, R. M., The use of dendritic cells in cancer immunotherapy. Curr. Opin. Immunol. 2003. 15: 138-147.
[8] Kaiser, A., Bercovici, N., Abastado, J. P. and Nardin A., Naive CD8+ T cells recruitment and proliferation are dependent on stage of dendritic cells maturation. Eur. J. Immunol. 2003. 33: 162-171.
[9] Santini, S. M., Lapenta, C., Logozzi, M., Parlato, S., Spada, M., Di Pucchio, T. and Belardelli, F., Type I interferon as a powerful adjuvant for monocyte-derived dendritic cell development and activity in vitro and in Hu-PBL-SCID mice. J. Exp. Med. 2000. 191: 1777-1788.
[10] Lapenta, C., Santini, S. M., Logozzi, M., Spada, M., Andreotti, M., Di Pucchio, T., Parlato, S., et al., Potent immune response against HIV-1 and protection from virus challenge in hu-PBL-SCID mice immunized with inactivated virus-pulsed dendritic cells generated in the presence of IFN-α. J. Exp. Med. 2003. 198: 361-367.
[11] Santodonato, L., D'Agostino, G., Nisini, R., Mariotti, S., Monque, D. M., Spada M., Lattanzi, L., et al., Monocyte-derived dendritic cells generated after a short-term culture with IFN-α and Granulocyte-Macrophage Colony-Stimulating Factor stimulate a potent Epstein-Barr virus-specific CD8+ T cell response. J. Immunol. 2003. 170: 5195-5202.
[12] Gabriele, L., Borghi, P., Rozera, C., Sestili, P., Andreotti, M., Guarini, A., Montefusco, E., et al., IFN-α promotes the rapid differentiation of monocytes from patients with chronic myeloid leucemia into activated dendritic cells tuned to undergo full maturation after LPS treatment. Blood 2004. 103: 980-987.
[13] Lapenta, C., Santini, S. M., Spada, M., Donati, S., Urbani, F., Accapezzato, D., Franceschini, D., et al., IFN-α-conditioned dendritic cells are highly efficient in inducing cross-priming CD8+ T cells against exogenous viral antigens. Eur. J. Immunol. 2006. 36: 2046-2060.
[14] Van den Eynde, B. J. and Morel, S., Differential processing of class-I-restricted epitopes by the standard proteasome and the immunoproteasome. Curr. Opin. Immunol. 2001. 13: 147-153.
[15] Macagno, A., Kuehn L., De Giuli, R. and Groettrup, M., Pronounced up-regulation of the PA28 α/β proteasome regulator but little increase in the steady-state content of immunoproteasome during dendritic cell maturation. Eur. J. Immunol. 2001. 31: 3271-3280.
[16] Gil-Torregrosa, B. C., Lennon-Dumenil, A. M., Kessler B., Guermonprez P., Ploegh H. L., Fruci D., Van Endert P. and Amigorena, S., Control of cross-presentation during dendritic cell maturation. Eur. J. Immunol. 2004. 34: 398-407.
[16 bis] Macagno, A., Gilliet, M., Sallusto, F., Lanzavecchia, A., Nestle, F. O. and Groettrup, M., Dendritic cells up-regulate immunoproteasomes and the proteasome regulator PA28 during maturation. Eur. J. Immunol. 1999. 29: 4037-4042.

1. A method of inducing a CD8⁺ T cell response to an antigenic peptide, comprising: culturing a monocytic cell in the presence of a Type I Interferon, Granulocyte-Mcrophage Clony-Simulating Factor (GM-CSF) and an antigen, to provide a cultured dendritic cell which presents said peptide complexed with an MHC class I molecule on its cell surface, and exposing the cultured dendritic cell to a population of naïve CD8+ T cells.

2. The method according to claim 1, wherein the interferon is interferon-alpha (IFNα).

3. The method according to claim 1, wherein the interferon is interferon-beta (IFNβ).

4. The method according to claim 1, wherein the cultured dendritic cell is CD123⁺ BDCA2⁺

5. The method according to claim 1, wherein the cultured dendritic cell is BDCA1⁻.

6. The method according to claim 1, wherein the cultured dendritic cell is characterised by up-regulation of at least one of the following: CD40, CD80 and DC86.

7. The method according to claim 1, wherein HSP70 recognition by the cultured dendritic cell is inhibited by the presence of an anti-HSP70 monoclonal antibody.

8. The method according to claim 1, wherein the cultured dendritic cell is capable of inducing a strong Th1 immune response, together with a CD8⁺ T cell response against the antigen.

9. The method according to claim 1, wherein the antigen is autologous or allogeneic.

10. The method according to claim 1, wherein the antigen is exogenous.

11. The method according to claim 1, wherein the cultured dendritic cell exhibits increased expression of the proteasome regulator sub unit PA28-alpha (PA28α).

12. The method according to claim 1, wherein the cultured dendritic cell shows increased expression of the catalytic sub units of its proteasome.

13. The method according to claim 1, wherein the method occurs in vitro and the expanded CD8⁺ T cells are introduced into the patient.

14. The method according to claim 1, wherein naïve CD8⁺ T cells have first been removed from the patient and are subsequently reintroduced to the same patient.

15. The method according to claim 1, wherein the patient is a human.

16. The method according to claim 1, wherein the MHC molecules are Class I HLA haplotypes.

17. The method according to claim 16, wherein the haplotype is HLA-A.

18. The method according to claim 17, wherein the haplotype is selected from: HLA-A1, HLA-A2, HLA-A3, HLA-A24, HLA-A29, HLA-A31 or HLA-A33.

19. The method according to claim 1, wherein the MHC molecules are Class I HLA haplotypes selected from: HLA-B, -C, -E, -F and -G.

20. The method according to claim 1, wherein the antigen is derived from a virus.

21. The method according to claim 20, wherein the virus is HIV.

22. The method according to claim 21, wherein the antigen is derived from the expression products of at least one of: gag, pol, env, and nef.

23. The method according to claim 1, wherein the antigen is a tumor-associated antigen (TAA).

24. The method according to claim 1, wherein the antigen is derived from Hepatitis viruses, Human Papillomavirus and Epstein Barr Virus

25. The method according to claim 23, wherein the tumor-associated antigen is selected from: the sub-dominant LMP-2 epitope of the Epstein Barr Virus (EBV), the NS3 peptide from Hepatitis C Virus (HCV), the E6 and E7 proteins from HPV (Human Papillomavirus).

26. The method according to claim 1, wherein the antigen is a tumoral antigen.

27. The method according to claim 1, wherein the tunoural antigen is selected from the group consisting of: those associated with cervical carcinoma, prostatic cancer, renal and lung cancer, and melanoma.

28. A vaccine for an antigen, comprising the dendritic cell as defined in claim 1 presenting an antigenic peptide, the vaccine being adapted for suitable administration to allow recognition of said antigen by a T cell receptor on a CD8⁺ T cell.

29. The vaccine of claim 28, wherein the vaccine is adapted to be administered intravenously, subdermally, intramusculuarly, transmucosally, intranodally, transdermally or in the form of a patch or spray.

30. A method of vaccination comprising administering the vaccine of claim 28 to a patient.

31. The method of vaccination of claim 30, the antigen is obtained from the patient by a blood sample or tissue extract, and contacted with the dendritic cell, thereby allowing the presentation of the antigen, or a fragment thereof, on the surface of the dendritic cell in complex with the MHC class 1 molecule, the dendritic cell, comprising said complex, being reintroduced into the patient, in the form of a vaccine.

32. A method of inducing a CD8⁺ T cell response to an antigenic peptide, comprising contacting a dendritic cell, which presents said peptide complexed with an MHC class I molecule, with a CD8+ T cell capable of recognizing said peptide-MHC class I complex, wherein the dendritic cell is obtainable by culturing a monocyte in the presence of Interferon and GM-CSF.

33. A method of inducing a CD8⁺ T cell response to an antigenic peptide, comprising contacting a dendritic cell with a CD8+ T cell,
the antigenic peptide being presented in a complex with an MHC class I molecule, or its equivalent, on the surface of the dendritic cell, and
the CD8+ T cell comprising a T cell receptor capable of recognizing said peptide-MHC class I complex, wherein
the dendritic cell is obtainable by culturing a monocyte in the presence of a type I interferon and GM-CSF.


SEQUENCE LISTING (4 sequences)

SEQ ID NO: 1 - DNA, 26 bp, artificial
  GH26 primer
gtgctgcaggtgtaaacttgtaccag

SEQ ID NO: 2 - DNA, 28 bp, artificial
  GH27 primer
gttgagatggcgacgatggcctaggcac

SEQ ID NO: 3 - DNA, 21 bp, artificial
  GAG 881 primer
ggtacatcaggccatatcacc

SEQ ID NO: 4 - DNA, 18 bp, artificial
  GAG 882 primer
ctctgatacatctggcca